SEQUENCE LISTING (23 sequences)

SEQ ID NO: 1 - protein, 210 aa, Borrelia burgdorferi
MKKNTLSAILMTLFLFISCNNSGKDGNASANSADESVKGPNLTEISKKITESNAVVLAVKEVETLLASINQLAKAIGKKIDQNGTLGDDGGQNGSLLAGAYAISTVIIEKLSTLKNVEELKEKITKAKDCSEKFAGKLKNEHASLGKKDATDDDAKKAILKTHGNTDKGAKELKDLSDSVESLVKAAKEMLTNSVKELTSPVVAESPKKP

SEQ ID NO: 2 - protein, 209 aa, Borrelia valaisiana
MKKNTLSAILMTLFLFISCNNSGGDTASTNPVDESAKGPNLTEISKKITDSNAIVLAVKEVETLLASINEIANKGIGKKINQNGLDNLTDHNGSLIAGAYVISTLITEKLNNLKNSEGLKEKIKKVKECSDKFTKKLTTSNGDLGKENVTDAHAQAAILKTNPTNDKGAKELGELFESVEILSKAAQEALTNSIAELTSPVVAENPKNP

SEQ ID NO: 3 - protein, 208 aa, Borrelia garinii
MKKNTLSAILMTLFLFISCNNSGGDTASTNPDESAKGPDLTVISKKITDSNAFVLAVKEVEALISSIDELANKAIGKVIHQNNGLNANAGQNGSLLAGAYAISTLITEKLSKLKNSEENKKIEEAKNHSEAFTNRLKGSHAQLGVAAATDDHAKEAILKSNPTKDKGAKELKDLSESVESLAKAAQEALANSVKELTNPVVAETPKKL

SEQ ID NO: 4 - protein, 210 aa, Borrelia afzelii
MKKNTLSAILMTLFLFISCNNSGKGGDSASTNPADESAKGPNLTEISKKITDSNAFVLAVKEVETLVSSIDELANKAIGKKIQQNGLGAEANRNESLLAGVHEISTLITEKLSKLKNSGELKAKIEDAKKCSEEFTNKLRVSHADLGKQGVNDDDAKKAILKTNADKTKGAEELGKLFKSVEGLVKAAQEALTNSVKELTSPVVAESPKK

SEQ ID NO: 5 - protein, 700 aa, Borrelia burgdorferi
MKKMLLIFSFFLIFLNGFPVSAREVDREKLKDFVNMDLEFVNYKGPYDSTNTYEQIVGIGEFLARPLTNSNSNSSYYGKYFINRFIDDQDKKASVDVFSIGSKSELDSILNLRRILTGYLIKSFDYDRSSAELIAKVITIYNAVYRGDLDYYKGFYIEAALKSLSKENAGLSRVYSQWAGKTQIFIPLKKDILSGNIESDIDIDSLVTDKVVAALLSENEAGVNFARDITDIQGETHKADQDKIDIELDNIHESDSNITETIENLRDQLEKATDEEHKKEIESQVDAKKKQKEELDKKAINLDKAQQKLDSAEDNLDVQRNTVREKIQEDINEINKEKNLPKPGDVSSPKVDKQLQIKESLEDLQEQLKETGDENQKREIEKQIEIKKSDEKLLKSKDDKASKDGKALDLDRELNSKASSKEKSKAKEEEITKSKSQKSLGDLNNDENLMMPEDQKLPEVKKLDSKKEFKPVSEVEKLDKIFKSNNNVGELSPLDKSSYKDIDSKEETVNKDVNLQKTKPQVKDQVTSLNEDLTTMSIDSSSPVFLEVIDPITNLGTLQLIDLNTGVRLKESTQQGIQRYGIYEREKDLVVIKMDSGKAKLQILDKLENLKVVSESNFEINKNSSLYVDSKMILVAVRDKDSSNDWRLAKFSPKNLDEFILSENKIMPFTSFSVRKNFIYLQDEFKSLVILDVNTLKKVK

SEQ ID NO: 6 - protein, 369 aa, Borrelia burgdorferi
MKKITSLVFILPLFVLITCKNTVSDEVIKNKLLNSIETLGKEFSDAFISSPFGGSLGTAAIDLKKSEVRNYFETISKGFKKIQEGFKKISFDFAEYDNAVHSINDIAKVFDWYLMEFVNDVEEAAKAADIDSDLVGQVVAVNPKIADSSSVKRFANWIQTLLRFSSVFGYDIYNKMFKNINIDSSNHRKHMQQVGNVKHVGKIFLKKENEDNHNCGTNNGSAENKAKDLVNSVSGEEILCLILRDIGGSPEHAKGKSPQSSENDPIEAAIGTNYAGDANSSSNCFVYDMPVNIAAAIVLRGMAKDGQFFAINGENDTVKTAVEIGVTKTLVALTTLLRGSVGEMIGRLDQIRKERLRFGTLVENNGYRY

SEQ ID NO: 7 - protein, 397 aa, Borrelia garinii
MKKISSAIFTIVFLVFINCKSDARKAINSIQTQKFTSFDGLLIDGFLSLKPNPKKSEVKDYFNSMAKTLNKTKDKLAKLISEKGGKTTEENNTDTAKEDNSTVNPVDNEINKIKDMIDKMIDAANTIVETVAETATETMGEVVEVKSSGNVATKADVKSVVEIAKGIKKIVGAAGIADKLKAEADKTTKPISEESNNKEAGKMFSGKQGDQGGKVIDSDVISPEIGGGANPMDINKAAEAVKNVSGEQILGAIIAAAKAIESGGKATTEGKNADEAKNPIEAAIGGNDDSNATAFTGNMEKDTQIAAAIVLRGMAKNGKFAVKMGRGPSADGNTIRALVKNAANKTVDALSQLVLKAINESLTKIAKTIKAGGEAANEAVNSNFPSVKFAEKHLEDK

SEQ ID NO: 8 - protein, 187 aa, Borrelia burgdorferi
MIKCNNKTFNNLLKLTILVNLLISCGLTGATKIKLESSAKAIVDEIDAIKKKAASMGVNFDAFKDKKTGSGVSENPFILEAKVRATTVAEKFVIAIEEEATKLKETGSSGEFSAMYDLMFEVSKPLQELGIQEMTKTVSMAAEENPPTTAQGVLEIAKKMREKLQRVHKKNQDTLKKKNTEDSTAKS

SEQ ID NO: 9 - protein, 175 aa, Borrelia garinii
MTKYIKNLLKLTLIVGLLVACSLTGKARLESSVKDITDEIDKAIKEAIADGVKLNELEENKTGAKKGGPQIRDAKIRVINLSVKFLKEIEEEANILKDNVGMNKVDKDQLLKDMYDLMLNAAGSLQKLGLQEMIKTVTQAAEKTPPTTVEGILMIANTIEDKLKKIKGKQETNKK

SEQ ID NO: 10 - protein, 170 aa, Borrelia afzelii
MIKYNKIILTLTLLASLLAACSLTGKARLESSVKDITNEIEKAIKEAEDAGVKTDAFTETQTGGKVGGSQIRAAKIRVADLTIKFLEATEEETITFKENGAGEEDFSGIYDLILNAAKAVEKIGMQGMKQAVEEAAKEKPKTTADGIIAIVKVMKAKVENIKEKQTKNQK

SEQ ID NO: 11 - protein, 187 aa, Borrelia burgdorferi
MKIGKLNSIVIALFFKLLVACSIGLVERTNAALESSSKDLKNKILKIKKEATGKGVLFEAFTGLKTGSKVTSGGLALREAKVQAIVETGKFLKIIEEEALKLKETGNSGQFLAMFDLMLEVVESLEDVGIIGLKARVLEESKNNPINTAERLLAAKAQIENQLKVVKEKQNIENGGEKKNNKSKKKK

SEQ ID NO: 12 - protein, 184 aa, Borrelia garinii
MKKFNLIIVALFVALLAACNFGLTGEVKAMLESSSDNVKNKILQIKEEAAKKGVNFKAFTGTATGSKVANGGSALREAKVQAINEVEKFLNTIEKEALILKKNGNSSQFLAMFDLMLEVTGSLDEIGIKGIKSSISEEAKSNPVNTAERLVEVKAKIENKLEGVKKRQKLDDEEKKISKSKKNK

SEQ ID NO: 13 - protein, 183 aa, Borrelia afzelii
MKKFNLIIEALFAILLTACNFGLMEETKIALESSSKDVKNKILQIKKDAEDKGVNFAAFTSSETGSKVTNGGLALREAKIQAINEVEKFLKRIEEEALKLKEHGNSGQFLELFDLLLEVLESLEPIGIKGLKDFISEEAKCNPISTSERLIEVKVQIENKMEEVKRKQNLNKERKSNKGKKKK

SEQ ID NO: 14 - protein, 178 aa, Borrelia burgdorferi
MEKYLSYIKKDDLDAIQLKLQELLASLHIFYSNLRGIHWNIKDTNFFVIHKKTQKLYEYIEKIIDIVAERSRMLGYDSEFRYSEFMKKSFIKELDIESTSNFLPSMESIVCSLTEILKNIFGMRKLIDTAGDYGTANIMDDIMSDLEKHLWMHKALLENCDCFCHDENESKCCECDAK

SEQ ID NO: 15 - protein, 176 aa, Borrelia afzelii
MEKYLSYIKKDDLEEIHSKLQELLASLHIFYSNLRGIHWNIKDTNFFVIHKKTQDLYEYIEKIIDIIAERSRMLGYDSEFRYSEFMKKSFIKELNIESTSNFSLSMQSIVSSLAEILKNIFGMRKLIDTACDYGTANLIDDIMSDLEKYLWMHKALLENCDCVCHDDNKCCECDIK

SEQ ID NO: 16 - protein, 177 aa, Borrelia burgdorferi
DGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYVLEGTLTAEKTTLVVKEGTVTLSKNISKSGEVSVELNDTDSSAATKKTAAWNSGTSTLTI

SEQ ID NO: 17 - protein, 274 aa, Borrelia garinii
MKKYLLGIGLILALIACKQNVSSLDEKNSVSVDLPGGMKVLVSKEKDKDGKYSLMATVEKLELKGTSDKSNGSGVLEGEKADKSKAKLTISQDLNQTTFEIFKEDGKTLVSRKVNSKDKSSTEEKFNDKGKLSEKVVTRANGTRLEYTEIKNDGSGKAKEVLKGFALEGTLTDGGETKLTVTEGTVTLSKNISKSGEITVALNDTETTPADKKTGEWKSDTSTLTISKNSQKPKQLVFTKENTITVQNYNRAGNALEGSPAEIKDLAELKAALK

SEQ ID NO: 18 - protein, 130 aa, Borrelia afzelii
QNVSSLDEKNSASVDLPGEMKVLVSKEKDKDGKYSLKATVDKIELKGTSDKDNGSGVLEGTKDDKSKAKLTIADDLSKTTFELFKEDGKTLVSRKVSSKDKTSTDEMFNEKGELSAKTMTRENGTKLEYT

SEQ ID NO: 19 - protein, 336 aa, Borrelia burgdorferi
MIINHNTSAINASRNNAINAANLSKTQEKLSSGYRINRASDDAAGMGVSGKINAQITGLSQASRNTSKAINFIQTTEGNLNEVEKVLVRMKELAVQSGNGTYSDADRGSIQIEIEQLTDEINRIADQAQYNQMHMLSNKSASQNVKTAEELGMQPAKINTPASLSGSQASWTLRVHVGANQDEAIAVNIYSANVANLFAGEGAQAAQAAPVQEGAQEEGAQQPTPATAPTQGGVNSPVNVTTTVDANTSLAKIENAIRMISDQRANLGAFQNRLESIKNSTEYAIENLKASYAQIKDATMTDEVVAATTNSILTQSAMAMIAQANQVPQYVLSLLR

SEQ ID NO: 20 - protein, 336 aa, Borrelia afzelii
MIINHNTSAINASRNNAINAANLSKTQEKLSSGYRINRASDDAAGMGVSGKINAQIRGLSQASRNTSKAINFIQTTEGNLNEVEKVLVRMKELAVQSGNGTYSDSDRGSIQIEIEQLTDEINRIADQAQYNQMHMLSNKSASQNVKTAEELGMQPAKINTPASLSGSQASWTLRVHVGANQDEAIAVNIYSANVANLFAGEGAQAAQAAPVQEGAQEEGAQQPTPATAPTQGGVNSPVNVTTTVDANTSLAKIENAIRMISDQRANLGAFQNRLESIKNSTEYAIENLKASYAQIKDATMTDEVVAATTNSILTQSAMAMIAQANQVPQYVLSLLR

SEQ ID NO: 21 - protein, 339 aa, Borrelia burgdorferi
MNKILLLILLESIVFLSCSGKGSLGSEIPKVSLIIDGTFDDKSFNESALNGVKKVKEEFKIELVLKESSSNSYLSDLEGLKDAGSDLIWLIGYRFSDVAKVAALQNPDMKYAIIDPIYSNDPIPANLVGMTFRAQEGAFLTGYIAARLSKTGKIGFLGGIEGEIVDAFRYGYEAGAKYANKDIKIFTQYIGSFADLEAGRSVATRMYSDEIDIIHHAAGLGGIGAIEVAKELGSGHYIIGVDEDQAYLAPDNVITSTTKDVGRALNIFTSNHLKTNTFEGGKLINYGLKEGVVGFVRNPKMISFELEKEIDNLSSKIINKEIIVPSNKESYEKFLKEFI

SEQ ID NO: 22 - protein, 361 aa, Borrelia garinii
MHLIYLNQSYKLLRYNFIWKGEQFMNKSLLLILFECIIFLSCGGKGSLENEIPKVSLIIDGTFDDKSFNESALNGIKKVKEEFKIEPVLKESSINSYLSDLEGLKDTGSNLIWLIGYKFSDVAKAVSLQNPEIKYAIIDPVYSDEPIPANLVGMTFRSQEGAFLTGYIAAKVSKTGKIGFLGGIEGEIVDSFRYGYEAGAKYANKDIKISAYYIGSFADVEAGRSVATKMYSDGIDIIHHAAGLGGIGAIEVAKELGSGHYIIGVDEDQSYLAPNNIITSATKDVGRSLNIFTSNYLKTNTFEGGRLINYGLKEGVVGFVKNPKMIPFELEKEIDNLSSKIINKEIIVPYNKESYEKFLKE

SEQ ID NO: 23 - protein, 339 aa, Borrelia afzelii
MNKLLLLILFEGVIFLSCSGKSGLESGIPKVSLVIDGTFDDKSFNESALNGVKKLKEEFEIELVLKESSTNSYLSDLEGLKDAGSNLIWLIGYKFSDVAKAVSLQNSEMKYAIIDPVYSNEPIPSNLVGMTFRAQEGAFLTGYIAAKVSKTGKIGFLGGIEGDIVDAFRYGYEAGAKYANKDIKIFSQYIGSFSDLEAGRSVATKMYSDGIDIIHHAASLAGIGAIEVAKELGSGHYIIGVDEDQSYLAPNNVITSTTKDVGRSLNLFTSNYLKTNTFEGGKLINYGLKEGVVGFVRNPKMIPFEVEKEIDSLSSKIINKEVIVPYNKESYEKFLKEFI

(19) United States
(12) Patent Application Publication
(10) Pub. No.: US 2015/0285798 A1
(43) Pub. Date: Oct. 8, 2015

(54) COMPOSITIONS AND METHODS FOR DETECTING, TREATING AND MONITORING ACTIVE BORRELIA INFECTION

(71) Applicant: Pharmasan Labs, Inc., Osceola, WI (US)

(72) Inventors: Chenggang Jin, Palo Alto, CA (US); Gottfried H. Kellermann, Osceola, WI (US)

(21) Appl. No.: 14/644,418

(22) Filed: Mar. 11, 2015

Related U.S. Application Data
(60) Provisional application No. 61/952,654, filed on Mar. 13, 2014.

(51) Int. Cl. G01N 33/569

(52) U.S. Cl. CPC G01N 33/56911; G01N 2333/96436; G01N 2333/54; G01N 2333/57; G01N 2469/20

(57) ABSTRACT

Disclosed herein are compositions and methods for detecting and treating active Borrelia infection in a subject.

### CLAIM OF PRIORITY

This application claims priority to U.S. Patent Application Ser. No. 61/952,654, filed on Mar. 13, 2014, the entire contents of which are hereby incorporated by reference.

### TECHNICAL FIELD

This document provides methods and materials related to compositions and methods for detecting and treating active Borrelia infection in a subject.

### BACKGROUND

Lyme disease, or Lyme borreliosis, is the most prevalent tick-borne disease of humans in the United States. The Centers for Disease Control and Prevention (CDC) reported nearly 32,500 new cases in 2011, though it is estimated that the actual number is 10-fold higher, making Lyme disease an epidemic larger than AIDS, West Nile Virus, and Avian Flu combined.

Lyme disease is transmitted by the bite of blacklegged ticks. Infection is caused by a spirochete bacterium of the Borrelia genus, e.g., Borrelia burgdorferi. The Borrelia infection results in an illness affecting various organs of the body. The clinical implications of Lyme disease or Borrelia infection can be seen in dermatologic, neurologic and rheumatologic manifestations. While there is significant variability in the presentation of Lyme disease, typical symptoms include fever, headache, fatigue, swollen lymph nodes, muscle and joint aches, and sometimes a characteristic bull-eye shaped skin rash called “erythema migrans.”

Diagnosis of Lyme disease or Borrelia infection is often based upon a physician's review of clinical symptoms and the patient's exposure risk in an area where the disease is endemic. Prompt diagnosis and treatment of Borrelia infection is the key to avoiding chronic Lyme disease and its deleterious effects. Early detection of Lyme disease can be difficult because the characteristic rash may not be present and the flu-like symptoms can be caused by many other factors which can confuse diagnosis.

For laboratory tests, the Centers for Disease Control (CDC) recommend a two-tiered approach consisting of an enzyme linked immunosorbent assay (ELISA) and a Western Blot (WB), both of which are serological assays that detect antibodies specific to an antigen of the Borrelia bacteria. The sensitivity of the two-tiered tests, however, is only about 30% in early Lyme disease and 50% in late Lyme disease. Moreover, the two-tiered tests report false-negative results for seronegative Lyme patients, consisting of about 30-50% of all Lyme patients.

### SUMMARY

Provided herein are methods and materials for detecting and treating active Borrelia infection in a subject. Active or acute Borrelia infection as used herein refers to the inflammatory condition in a subject that is caused by exposure to one or more Borrelia antigens within the prior 6 months, and typically within the prior 3 months, e.g., within the prior 90 days, within the prior 60 days, or within the prior 30 days. The present invention is based in part on the development of a novel highly sensitive Lyme disease-specific enzyme-linked immunosorbent spot assay (Lyme ELISpot) that is capable of detecting Borrelia antigen-specific CD8 effector T cells at single cell resolution. Active Borrelia infection can be distinguished from latent (also referred to asdormant, or chronic herein) Borrelia infection by measuring one or more proteins secreted by Borrelia antigen-specific CD8 effector T cells, e.g., granzyme B, perforin, or granulysin, and determining the ratio of Lyme antigen-specific CD8 effector T cells to peripheral blood mononuclear cells (PBMCs). Latent infection as used herein refers to the inflammatory condition in a subject that is caused by exposure to one or more Borrelia antigens in a time period greater than 6 months earlier, and typically a year or more earlier. High sensitivity of the Lyme ELISpot assay is ensured by a combination of serum-free medium, purified recombinant Borrelia antigens, and/or co-stimulation by Interleukin-7 (IL-7). Provided herein are also methods of treating active Borrelia infection if the ratio of Lyme antigen-specific CD8 effector T cells to PBMCs is above a reference level, e.g., the ratio of Lyme antigen-specific CD8 effector T cells to PBMCs in a healthy subject. Also provided herein are kits that comprise (1) a solid phase support, e.g., a particle or a microwell of a microplate, which is coated with one or more capture antibodies specific for one or more proteins secreted by Lyme antigen-specific CD8 effector T cells, e.g., granzyme B, perforin, or granulysin; and (2) a composition comprising one or more Borrelia antigen polypeptides or antigenic fragments thereof, e.g., one or more of NapA, VlsE, DbpA, DbpB, OspC, OspA, OspB, P100, P41, P66, BmpA, BmpB, BmpC, Bgp, and Fbp.

In one aspect, methods for detecting active Borrelia infection in a subject are provided. These method include the steps of (1) providing peripheral blood mononuclear cells (PBMCs) of the subject; (2) incubating the PBMCs in a serum-free medium with one or more Borrelia antigens for about 18 to about 72 hours; and (3) determining the ratio of Borrelia antigen-specific CD8 effector T cells to PBMCs by measuring the level of one or more proteins secreted by Borrelia antigen-specific CD8 effector T cells, e.g., granzyme B, perforin, or granulysin. If the ratio of Borrelia antigen-specific CD8 effector T cells to PBMCs is above a reference level, e.g., the ratio of Borrelia antigen-specific CD8 effector T cells to PBMCs in a healthy subject, it is indicative of active Borrelia infection in the subject. Incubation of PBMC with Borrelia antigens for about 18 to about 72 hours ensures detection of only Borrelia antigen-specific CD8 effector/effector memory T cells that had recently encountered Borrelia infection within the prior 6 months, but does not reactivate CD8 central memory T cells. The serum-free medium can also contain interleukin-7.

Also provided herein are methods of treating active Borrelia Infection in a subject. Such methods include (1) providing peripheral blood mononuclear cells (PBMCs) of the subject, (2) incubating the PBMCs in a serum-free medium with one or more Borrelia antigens for about 18 to about 72 hours; (3) determining the ratio of Borrelia antigen-specific CD8 effector T cells to PBMCs by measuring the level of one or more proteins secreted by Borrelia antigen-specific CD8 effector T cells, e.g., granzyme B, perforin, or granulysin, (4) if the ratio of Borrelia antigen-specific CD8 effector T cells to PBMCs is above a reference level, administering to the subject a treatment suitable for treating active Borrelia infection. The reference level can be the ratio of Lyme antigen-specific CD8 effector T cells to PBMCs in a healthy subject. Treatments for active Borrelia infection can include administration of one or more antibiotics, e.g., doxycycline, amoxicillin, cefuroxime axetil, ceftriaxone, cefotaxime, penicillin, clarithromycin, erythromycin, and azithromycin, by an oral, intravenous or parenteral route.

The one or more Borrelia antigens are polypeptides or proteins derived from or exhibiting sequence similarity to polypeptides or proteins derived from one or more pathogenic species of Borrelia: e.g., Borrelia burgdorferi sensu stricto, Borrelia afzelii, Borrelia garinii, Borrelia valaisiana, Borrelia bissettii, Borrelia lusitaniae, and Borrelia spielmanii. The Borrelia antigens can be native, recombinant, or synthetic.

The one or more proteins secreted by the Borrelia antigen-specific CD8 effector T cells can be measured by performing a bioassay, an immunoassay, a flow cytometry, or a radioimmunoassay (RIA). Preferably, an enzyme-linked immunosorbent spot (ELISpot) assay is performed to measure the level of one or more proteins secreted by the Borrelia antigen-specific CD8 effector T cells.

In some embodiments, the methods of detecting active Borrelia infection described herein further comprises measuring one or more cytokines secreted by the PBMCs, e.g., IL-17, IFN-γ, IL-2, IL-3, IL-4, IL-5, IL-6, IL-9, IL-10, IL-13, IL-21, IL-22, IL-25, IL-31, TNF-α, TNF-β, and GM-CSF.

In some embodiments, a latent or dormant Borrelia infection in a subject can be detected by determining the ratio of granzyme B-secreting CD8 T cells to PBMCs and the ratio of IFN-γ-secreting CD8 T cells to PBMCs, and comparing these two ratios. If the ratio of IFN-γ-secreting CD8 T cells to PBMCs is greater than the ratio of granzyme B-secreting CD8 T cells to PBMCs, the subject is likely to have a latent or dormant Borrelia infection since both CD8 central memory T cells and CD8 effector T cells are able to secret IFN-γ.

The Borrelia antigens used to stimulate peripheral blood mononuclear cells can be a selection of one or more of the following polypeptides or antigenic fragments thereof: Variable major protein-like gene E (VlsE), Neutrophil activating protein (NapA), Decorin-binding protein A (DbpA), Decorin-binding protein B (DbpB), Outer surface protein C (OspC), Outer surface protein A (OspA), Outer surface protein B (OspB), P100, P41, P66, Borrelia membrane protein A (BmpA), Borrelia membrane protein B (BmpB), Borrelia membrane protein C (BmpC), Borrelia glycosaminoglycan-binding protein (Bgp), and Fibronectin-binding protein (Fbp).

In some embodiments, the Borrelia antigen selection is a mixture of a NapA polypeptide or an antigenic fragment thereof, with one or more polypeptides or antigenic fragments thereof selected from VlsE, DbpA, DbpB, OspC, OspA, OspB, P100, P41, p66, BmpA, BmpB, BmpC, Bgp, and Fbp. In some embodiments, the Borrelia antigen selection is a mixture of a NapA polypeptide or an antigenic fragment thereof, a DbpA polypeptide or an antigenic fragment thereof, an OspC polypeptide or an antigenic fragment thereof, a P100 polypeptide or an antigenic fragment thereof, and a VlsE polypeptide or an antigenic fragment thereof. In some embodiments, the Borrelia antigen selection is a mixture of a NapA polypeptide or an antigenic fragment thereof, an OspC polypeptide or an antigenic fragment thereof, and a VlsE polypeptide or an antigenic fragment thereof.

In some embodiments, the Borrelia antigen selection is a mixture of an OspA polypeptide or an antigenic fragment thereof, with one or more polypeptides or antigenic fragments thereof selected from NapA, VlsE, DbpA, DbpB, OspC, OspB, P100, P41, p66, BmpA, BmpB, BmpC, Bgp, and Fbp. In some embodiments, the Borrelia antigen selection is a mixture of an OspA polypeptide or an antigenic fragment thereof, a DbpA polypeptide or an antigenic fragment thereof, an OspC polypeptide or an antigenic fragment thereof, a P100 polypeptide or an antigenic fragment thereof, and a VlsE polypeptide or an antigenic fragment thereof. In some embodiments, the Borrelia antigen selection is a mixture of an OspA polypeptide or an antigenic fragment thereof, an OspC polypeptide or an antigenic fragment thereof, and a VlsE polypeptide or an antigenic fragment thereof.

In some embodiments, the Borrelia antigen selection is a mixture of a NapA polypeptide or an antigenic fragment thereof, a DbpA polypeptide or an antigenic fragment thereof, an OspC polypeptide or an antigenic fragment thereof, a P100 polypeptide or an antigenic fragment thereof, a VlsE polypeptide or an antigenic fragment thereof, and an OspA polypeptide or an antigenic fragment thereof. In some embodiments, the Borrelia antigen selection is a mixture of a DbpA polypeptide or an antigenic fragment thereof, an OspC polypeptide or an antigenic fragment thereof, a P100 polypeptide or an antigenic fragment thereof, and a VlsE polypeptide or an antigenic fragment thereof. In some embodiments, the Borrelia antigen selection is a mixture of an OspC polypeptide or an antigenic fragment thereof and a VlsE polypeptide or an antigenic fragment thereof.

In some embodiments, an OspC polypeptide comprises an amino acid sequence having at least 80% sequence identity to any one of SEQ ID NOs:1-4, or to an antigenic fragment thereof. For example, an OspC polypeptide can have an amino acid sequence of any one of SEQ ID NOs:1-4.

In some embodiments, a P100 polypeptide comprises an amino acid sequence having at least 80% sequence identity to SEQ ID NO:5, or to an antigenic fragment thereof. For example, a P100 polypeptide can have an amino acid sequence of SEQ ID NO:5.

In some embodiments, a VlsE polypeptide comprises an amino acid sequence having at least 80% sequence identity to any one of SEQ ID NOs:6-7, or to an antigenic fragment thereof. For example, a VlsE polypeptide can have an amino acid sequence of any one of SEQ ID NOs:6-7.

In some embodiments, a DbpA polypeptide comprises an amino acid sequence having at least 80% sequence identity to any one of SEQ ID NOs:8-10, or to an antigenic fragment thereof. For example, a DbpA polypeptide can have an amino acid sequence of any one of SEQ ID NOs:8-10.

In some embodiments, a DbpB polypeptide comprises an amino acid sequence having at least 80% sequence identity to any one of SEQ ID NOs:11-13, or to an antigenic fragment thereof. For example, a DbpB polypeptide can have an amino acid sequence of any one of SEQ ID NOs:11-13.

In some embodiments, a NapA polypeptide comprises an amino acid sequence having at least 80% sequence identity to any one of SEQ ID NOs:14-15, or to an antigenic fragment thereof. For example, a NapA polypeptide can have an amino acid sequence of any one of SEQ ID NOs:14-15.

In some embodiments, an OspA polypeptide comprises an amino acid sequence having at least 80% sequence identity to any one of SEQ ID NOs:16-18, or to an antigenic fragment thereof. For example, an OspA polypeptide can have an amino acid sequence of any one of SEQ ID NOs:16-18.

In some embodiments, a P41 polypeptide comprises an amino acid sequence having at least 80% sequence identity to any one of SEQ ID NOs:19-20, or to an antigenic fragment thereof. For example, a P41 polypeptide has an amino acid sequence of any one of SEQ ID NOs:19-20.

In some embodiments, a BmpA polypeptide comprises an amino acid sequence having at least 80% sequence identity to any one of SEQ ID NOs:21-23, or to an antigenic fragment thereof. For example, a BmpA polypeptide has an amino acid sequence of any one of SEQ ID NOs:21-23.

In some embodiments, the method for detecting and treating active Borrelia infection in a subject can also include observing a Lyme disease related symptom in a subject, e.g., a tick bite, erythema migrans, skin lesion, pain, fever, headache, and/or swelling, or measuring a Borrelia antigen specific antibody in a blood sample of the subject using Western blot or enzyme-linked immunosorbent assay (ELISA).

In yet another aspect, a kit for detecting active Borrelia infection is provided. The kit includes a solid phase support, e.g., a particle or a microwell of a microplate, which is coated with one or more capture antibodies specific for one or more proteins secreted by Lyme antigen-specific CD8 effector T cells; and a composition comprising one or more Borrelia antigen polypeptides. The kit can also include instructions for use and other reagents such as serum-free medium; IL-7; a positive control, e.g., phytohaemagglutinin (PHA); a detection antibody; a chromogenic, fluorogenic, or electrochemiluminescent substrate; buffers and antimicrobial agents. The capture and detection antibodies can be monoclonal or polyclonal antibodies that bind to different epitopes on the cytokine. The detection antibody can be any detectably labeled antibody, for example, an antibody tagged with a fluorescent dye, an enzyme-conjugated antibody, or an antibody conjugated with one member of a specific binding pair, e.g., an antibody conjugated with biotin or streptavidin. For example, if a biotinylated detection antibody is included in the kit, the kit also includes enzyme-conjugated streptavidin.

The term “Borrelia antigen” used herein refers to polypeptides or proteins derived from or exhibiting sequence similarity to polypeptides or proteins derived from one or more pathogenic species of Borrelia: e.g., Borrelia burgdorferi sensu stricto, Borrelia afzelii, Borrelia garinii, Borrelia valaisiana, Borrelia bissettii, Borrelia lusitaniae, and Borrelia spielmanii. The terms “polypeptide” and “protein” are used interchangeably herein and refer to any chain of amino acids, regardless of length or post-translational modification (e.g., glycosylation or phosphorylation).

The term “antigenic fragment” used herein refers to a portion of a polypeptide capable of binding to a major histocompatibility complex (MHC) and being presented to a T-cell receptor.

The term “mixture” or “antigen mixture” used herein refers to a composition comprising at least two Borrelia antigen polypeptides or proteins.

As used herein, the term “percent sequence identity” refers to the degree of identity between any given query sequence and a subject sequence. Percentage of “sequence identity” is determined by comparing two optimally aligned sequences over a comparison window, where the fragment of the amino acid sequence in the comparison window may comprise additions or deletions (e.g., gaps or overhangs) as compared to the reference sequence (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity. The output is the percent identity of the subject sequence with respect to the query sequence. It is noted that a query nucleotide or amino acid sequence that aligns with a subject sequence can result in many different lengths, with each length having its own percent identity.

As used herein, a “subject” is an animal, e.g., a mammal, e.g., a human, monkey, dog, cat, horse, cow, pig, goat, rabbit, or mouse.

As used herein, the term “treat” or “treatment” is defined as the application or administration of a treatment regimen, e.g., a therapeutic agent or modality, to a subject. The treatment can be to cure, heal, alleviate, relieve, alter, remedy, ameliorate, palliate, improve or affect Lyme disease or symptoms associated with Lyme disease.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 shows representative Granzyme B Lyme ELISpot assay results of a Lyme patient using the various Borrelia antigens as shown. Tests were run in triplicate for each condition. Serum-free medium alone serves as a negative control.

FIG. 2 shows representative IL-17 Lyme ELISpot assay results of a Lyme patient using Borrelia antigen NapA or Borrelia Antigen mix 1. Tests were run in triplicate for each condition. Serum-free medium alone serves as a negative control, and phytohaemagglutinin (PHA) serves as a positive control.

FIG. 3 shows representative IFN-γ Lyme ELISpot assay results of a Lyme patient using Borrelia Antigen mix 1 or 2. While both the IFN-γ Lyme ELISpot assay with IL-7 and the IFN-γ Lyme ELISpot assay without IL-7 reported positive results, the addition of IL-7 dramatically amplified the signals of Borrelia antigen-positive cells.

FIG. 4 shows representative IFN-γ Lyme ELISpot results of a healthy donor using Borrelia Antigen mix 1 or 2. Both IFN-γ Lyme ELISpot assays (with or without IL-7) reported negative results. Serum-free medium alone serves as a negative control, and PHA serves as a positive control.

FIG. 5 contains amino acid sequences for Borrelia antigens set forth as SEQ ID NOs:1-23.

### DETAILED DESCRIPTION

Borrelia infection can be cured with antibiotic treatment alone if treatment begins early in the course of illness. Unfortunately only a small fraction of Borrelia infection is being treated in a timely manner due to equivocal clinical manifestations, inaccurate tests, and underreporting. Undiagnosed and untreated Borrelia infection can result in the development of a chronic Lyme infection or late stage Lyme diseases such as chronic Lyme arthritis or chronic Lyme neuroborreliosis, which can have devastating consequences in certain cases.

Provided herein are methods and materials for detecting and treating active Borrelia infection in a subject. The present invention is based in part on the development of a novel highly sensitive Lyme disease-specific enzyme-linked immunosorbent spot assay (Lyme ELISpot) that is capable of detecting Borrelia antigen-specific CD8 effector T cells at single cell resolution. Active Borrelia infection is distinguished from latent or dormant Borrelia infection by measuring one or more proteins secreted by Borrelia antigen-specific CD8 effector T cells, e.g., granzyme B, perforin, granulysin, and determining the ratio of Lyme antigen-specific CD8 effector T cells to peripheral blood mononuclear cells (PBMCs). High sensitivity of the Lyme ELISpot assay is ensured by a combination of serum-free medium, purified recombinant Borrelia antigens, and/or co-stimulation by Interleukin-7 (IL-7). Provided herein are also methods of treating active Borrelia infection if the ratio of Lyme antigen-specific CD8 effector T cells to PBMCs is above a reference level, e.g., the ratio of Lyme antigen-specific CD8 effector T cells to PBMCs in a healthy subject. Also provided herein are kits that comprise (1) a solid phase support, e.g., a particle or a microwell of a microplate, which is coated with one or more capture antibodies specific for one or more proteins secreted by Lyme antigen-specific CD8 effector T cells, e.g., granzyme B, perforin, granulysin; and (2) a composition comprising one or more Borrelia antigen polypeptides or antigenic fragments thereof, e.g., one or more of NapA, VlsE, DbpA, DbpB, OspC, OspA, OspB, P100, P41, P66, BmpA, BmpB, BmpC, Bgp, and Fbp.

### Methods for Detecting Active Borrelia Infection

Both humoral and cellular immune responses develop in Borrelia infection. T cell-mediated cytokine secretion occurs much earlier in the disease progression than B cell-mediated antibody response. Assessment of both the function and the frequency of Borrelia-specific T cells can help evaluate the cellular immune response to, and diagnosis of Borrelia infection (Dressler, F., et al., Ann. Intern. Med. 115: 533-539, 1991; Gross, D. M., et al., Science 281: 703-706, 1998).

CD8 T cells participate in defense against infection caused by pathogens such as viruses, bacteria, and protozoans (Wong, P.; Pamer, E. G., Annu Rev. Immunol. 21: 29-70, 2003). In response to a specific pathogen, naive CD8 T cells undergo clonal expansion, synthesis and storage of cytotoxins such as granzyme B and perforin, and acquisition of the ability to kill, and become CD8 effector T cells (also known as cytotoxic T cells) (Wherry, E. J. et al., J. Virol. 78: 5535-5545, 2004). The CD8 effector T cells can also secrete IFN-γ, TNF, and other effector molecules. The CD8 effector cells release these effector molecules towards the target cell and exert cytotoxic effects. For example, perforin punches holes in the cell membrane of target cells and granzyme B penetrate into target cells through these holes and induce apoptosis by activating the caspase pathways (Catalfamo, M., Curr. Opin. Immunol. 15: 522-527, 2003; Trapani, J. A., Nat. Rev. Immunol. 2: 735-747, 2002; Trapani, J. A., Curr. Opin. Immunol. 15: 533-543, 2003). CD8 T cells can also kill utilizing Fas-FasL interactions (Henkart, P. A., Immunity 1: 343-346, 1994; van den Brink, M. R., Nat. Rev. Immunol. 2: 273-281, 2002).

CD8 effector T cells are short lived. As soon as the pathogen is cleared, or if a pathogen persists, as soon as its replication stops, CD8 effector T cells stop expressing cytotoxins granzyme B and perforin and become quiescent memory T cells (Nowacki T M, Cells 1: 35-50, 2012). Upon renewed antigen encounter, the memory T cells are capable of secreting cytokines such as IFN-γ, but needs about three days to replenish their granules with granzyme B and perforin and to reacquire the ability to kill (Nowacki T M, Cells 1: 35-50, 2012). In this reactivated state, they are referred to as effector memory T cells. Both CD8 effector T cells and CD8 effector memory T cells can kill target cells utilizing perforin and granzyme B. Granzyme B has been used to detect active Cytomegalovirus infection in patients (Nowacki T M, Cells 1: 35-50, 2012). In the absence of continuous cytomegalovirus antigen stimulation, the CD8 effector memory T cells acquire a resting phenotype, and within about 30 days lose perforin and granzyme B (Nowacki T M, Cells 1: 35-50, 2012). Such resting CD8 cells are called central memory T cells. These central memory T cells are still able to secrete IFN-γ (Nowacki T M, Cells 1: 35-50, 2012). Therefore, the ability to instantaneously engage in secretion of perforin and granzyme B is a characteristic of CD8 effector T cells and CD8 effector memory T cells that had recently encountered a particular antigen exposure in vivo (Nowacki T M, Cells 1: 35-50, 2012). In contrast, the central memory T cells that had encountered antigen in the distant prior are not capable of instantaneous secretion of perforin and granzyme B, but can re-acquire this ability within three days (Kaech, S. M., Nat. Immunol. 2: 415-422, 2001).

Disclosed herein are methods of detecting active Borrelia infection in a subject, e.g., a human being, by (1) providing peripheral blood mononuclear cells (PBMCs) of the subject; (2) incubating the PBMCs in a serum-free medium with one or more Borrelia antigens, e.g., for about 18 to about 72 hours, e.g., about 18 to about 48 hours, about 20 to about 24 hours; and (3) determining the ratio of Borrelia antigen-specific CD8 effector T cells to PBMCs by measuring the level of one or more proteins secreted by Borrelia antigen-specific CD8 effector T cells, e.g., granzyme B, perforin. If the ratio of Borrelia antigen-specific CD8 effector T cells to PBMCs is above a reference level, e.g., the ratio of Borrelia antigen-specific CD8 effector T cells to PBMCs in a healthy subject, it is indicative of active Borrelia infection in the subject. Incubation of PBMC with Borrelia antigens for about 18 to about 72 hours ensures detection of only Borrelia antigen-specific CD8 effector T cells or CD8 effector memory T cells that had recently encountered Borrelia infection, but does not reactivate CD8 central memory T cells. High sensitivity of the Lyme ELISpot assay is ensured by a combination of serum-free medium, purified recombinant Borrelia antigens, and/or co-stimulation by Interleukin-7 (IL-7).

Th17 cells, a new subset of T helper cells that secrete IL-17, play a role in the pathogenesis of Lyme arthritis (Codolo G, et al., Arthritis Rheum. 58(11):3609-17, 2008; Burchill Mass., Infect Immun 71:3437-3442, 2003). In patients with Lyme arthritis, Neutrophil-activating protein A (NapA) of Borrelia was shown to stimulate monocyte production of IL-23, IL-6, IL-1β, and TGFβ, key cytokines for Th17 cell differentiation and drive Th17 cell inflammation (Codolo G, et al., Arthritis Rheum. 58(11):3609-17, 2008). T helper cells from the synovial fluid of patients with Lyme arthritis produce IL-17 in response to NapA (Codolo G, et al., Arthritis Rheum. 58(11):3609-17, 2008). Interestingly, IL-17 production cannot be detected in the peripheral blood T cells from the same patients with Lyme arthritis by ELISpot assays (see pages 3515-3516 of Codolo G, et al., Arthritis Rheum. 58(11):3609-3617, 2008).

In some embodiments, the methods of detecting active Borrelia infection described herein further comprises measuring one or more cytokines secreted by the PBMCs, e.g., IL-17, IFN-γ, IL-2, IL-3, IL-4, IL-5, IL-6, IL-9, IL-10, IL-13, IL-21, IL-22, IL-25, IL-31, TNF-α, TNF-β, and GM-CSF.

In some embodiments, a latent or dormant Borrelia infection in a subject can be detected by determining the ratio of granzyme B-secreting CD8 T cells to PBMCs and the ratio of IFN-γ-secreting CD8 T cells to PBMCs, and comparing these two ratios. If the ratio of IFN-γ-secreting CD8 T cells to PBMCs is greater than the ratio of granzyme B-secreting CD8 T cells to PBMCs, the subject is likely to have a latent or dormant Borrelia infection since both CD8 central memory T cells and CD8 effector T cells are able to secret IFN-γ.

Peripheral blood mononuclear cells (PBMCs) can be obtained from a subject's whole blood. Suitable methods for obtaining PBMCs can be used. For example, a whole peripheral blood sample can be obtained from a subject having or suspected of having Lyme disease (e.g., experiencing symptoms associated with Lyme disease). The PBMCs, which include lymphocytes, macrophages, and other white blood cells, can be isolated from whole peripheral blood by any appropriate method (e.g., centrifugation or density gradient). In some embodiments, PBMCs can be isolated from the whole blood by centrifugation, washed, and then suspended in medium with antibiotic, e.g., CTL-Test Medium (Cellular Technology Limited), or RPMI medium (Gibco, Grand Island, N.Y.) with penicillin/streptomycin and 1% glutamine.

PBMCs can be cultured alone or in the presence of Borrelia antigens or a positive control antigen (e.g., phytohemagglutinin) Cell-free supernatants can be collected from stimulated and non-stimulated or control cell cultures for analysis. The proteins secreted by Borrelia antigen-specific CD8 effector T cells that can be measured include granzyme B, perforin, granulysin, IL-17, IFN-γ, IL-2, IL-3, IL-4, IL-5, IL-6, IL-9, IL-10, IL-13, IL-21, IL-22, IL-25, IL-31, TNF-α, TNF-β, and GM-CSF. In some embodiments, one or more of granzyme B, perforin, and granulysin can be measured as the proteins secreted by Borrelia antigen-specific CD8 effector T cells. Other proteins that can be measured include any of the interleukins (IL), tumor necrosis factors (TNF), interferons (IFN), colony stimulating factors (CSF), leukemia inhibitory factor (LIF), transforming growth factors (TGF), or epidermal growth factor (EGF).

Methods of measuring the one or more proteins secreted by Borrelia antigen-specific CD8 effector T cells can include, for example, a bioassay, an immunoassay, a flow cytometry, a radioimmunoassay (RIA), an enzyme-linked immunosorbent assay (ELISA), an enzyme-linked immunosorbent spot assay (ELISpot), or measurement of messenger RNA levels. In general, immunoassays involve using a monoclonal antibody to the cytokine of interest to specifically bind and detect the cytokine Immunoassays are well-known in the art and can include both competitive assays and immunometric assays (see Ausubel et al., Current Protocols in Molecular Biology, 11.2.1-11.2.19 (1993); Laboratory Techniques in Biochemistry and Molecular Biology, Work et al., ed. (1978)).

The enzyme-linked immunosorbent spot (ELISpot) assay allows visualization of a secretory product of individual activated or responding cells. Each spot developed in the assay represents a single reactive cell. Thus, the ELISpot assay can accurately detect, measure, and perform functional analysis of low-frequency immune cells at single cell resolution.

The ELISpot assays can employ two high-affinity antibodies directed against different epitopes on the same secreted protein. More specifically, a microtiter plate can be coated with a capture antibody specific to a first epitope on the secreted protein. PBMCs can be plated on the coated microtiter plate at a desired density in serum-free medium alone (negative control), or in serum-free medium with the addition of one or more Borrelia antigens or PHA (positive control), and incubated, e.g., in a humidified 37° C. CO₂ incubator, e.g., for about 18 to about 72 hours, e.g., about 18 to about 48 hours, about 20 to about 24 hours. In some embodiments, IL-7 can be added to the serum-free medium and used for all conditions. The incubation time should be sufficient to permit Borrelia antigen-specific CD8 effector T cells to secrete proteins such as perforin and granzyme B, but avoid prolonged duration to reactivate the central memory T cells. During the incubation period, the proteins secreted by Borrelia antigen-specific CD8 effector T cells are captured locally by the capture antibodies immobilized on the plate.

After washing the wells to remove cells, debris, and media components, a detection antibody specific for a distinct second epitope on the protein can be added to detect the captured proteins. The detection antibody can be any detectably labeled antibody, for example, an antibody tagged with a fluorescent dye, an enzyme-conjugated antibody, or an antibody conjugated with one member of a specific binding pair, e.g., an antibody conjugated with biotin or streptavidin. For example, if a biotinylated detection antibody is employed, enzyme-conjugated streptavidins can also be used to detect the cytokine-antibody complexes. After washing to remove any unbound detection antibody, the secreted protein-antibody complexes captured in the wells can then be visualized using a chromogenic, fluorogenic, or electrochemiluminescent substrate of the enzyme. Because the proteins are captured locally upon secretion, each visible spot represents an individual Borrelia antigen-specific CD8 effector T cell, offering single cell resolution for the test. The spots can be counted manually (e.g., with a dissecting microscope) or using an automated analyzer, e.g., CTL-ImmunoSpot Analyzer (Cellular Technology Limited).

Any composition of Borrelia antigens described herein can be used for the Lyme ELISpot assay. Titration experiments can be performed to determine suitable concentrations for the Borrelia antigens.

In some embodiments, Lyme ELISpot assays can be used in parallel with other methods of diagnosing Lyme disease, including subjective (e.g., self-report of symptoms) and objective measurements of Lyme disease symptoms. For example, the methods provided herein can be used in parallel with clinical observations of, or a subject's self-reporting of, tick bite, erythema migrans (or bull-eye shaped rash), skin lesion, pain, fever, headache, swelling, or other symptoms associated with Lyme disease. In some embodiments, Lyme ELISpot assays can be used in parallel or mixture with Western blot analysis or other serological assays for the presence of Borrelia-specific antibodies.

In some embodiments, the assays used to differentiate active Borrelia infection from a latent Borrelia infection include one or more of the following: Granzyme B or Perforin Lyme ELISpot assay, IL-17 Lyme ELISpot assay, IFN-γ Lyme ELISpot assay, B cell ELISpot assay, Bio-Plex Pro human Th17 cytokine assays (BIO-RAD), and/or flow cytometry-based detection of Borrelia-specific T cells by using Borrelia peptide-MHC Dextramer (IMMUDEX) (See Oosting et al., Eur. J. Immunol. 41: 172-181, 2011; Henningsson et al., Journal of Neuroinflammation 8:36, 2011; Codolo et al., Arthritis & Rheumatism 58 (11): 3609-3617, 2008).

### Methods of Treating Borrelia Infection

Also provided herein are methods of treating active Borrelia Infection in a subject. Such methods include (1) providing peripheral blood mononuclear cells (PBMCs) of the subject, (2) incubating the PBMCs in a serum-free medium with one or more Borrelia antigens, e.g., for about 18 to about 72 hours, e.g., about 18 to about 48 hours, about 20 to about 24 hours; (3) determining the ratio of Borrelia antigen-specific CD8 effector T cells to PBMCs by measuring the level of one or more proteins secreted by Borrelia antigen-specific CD8 effector T cells, e.g., granzyme B, perforin, or granulysin, (4) if the ratio of Borrelia antigen-specific CD8 effector T cells to PBMCs is above a reference level, administering to the subject a treatment suitable for treating active Borrelia infection. The reference level can be the ratio of Lyme antigen-specific CD8 effector T cells to PBMCs in a healthy subject.

As used herein, the term “treat” or “treatment” is defined as the application or administration of a treatment regimen, e.g., a therapeutic agent or modality, to a subject, e.g., a patient. The subject can be a patient having Lyme disease or a symptom of Lyme disease, or at risk of developing Borrelia Infection or Lyme disease (e.g., frequently outdoors, living in a tick infested area). The treatment can be to cure, heal, alleviate, relieve, alter, remedy, ameliorate, palliate, improve or affect Borrelia Infection or Lyme disease or symptoms associated with Borrelia Infection or Lyme disease.

Treatments for active Borrelia infection include, without limitation, administration of one or more antibiotics, e.g., doxycycline, amoxicillin, cefuroxime axetil, ceftriaxone, cefotaxime, penicillin, clarithromycin, erythromycin, and azithromycin, by an oral, intravenous or parenteral route. For some subjects with active Borrelia infection without neurological or cardiac manifestations, oral administration of antibiotics such as doxycycline, amoxicillin, or cefuroxime axetil for two to three weeks can usually cure the infection. For example, treatment with doxycycline (100 mg twice per day), amoxicillin (500 mg 3 times per day), or cefuroxime axetil (500 mg twice per day) for 10-21 days is recommended by the CDC for adult patients with early localized or early disseminated Lyme disease associated with erythema migrans, in the absence of specific neurologic manifestations or advanced atrioventricular heart block (Wormser G P, Clinical Infectious Diseases 2006; 43:1089-1134).

Patients with certain neurological or cardiac forms of illness may require intravenous treatment with drugs such as ceftriaxone or penicillin. For example, the use of ceftriaxone (2 g once per day intravenously for 14 days; range, 10-28 days) in early Lyme disease is recommended for adult patients with acute neurologic disease manifested by meningitis or radiculopathy (Wormser G P, Clinical Infectious Diseases 2006; 43:1089-1134). Parenteral therapy with cefotaxime (2 g intravenously every 8 h) or penicillin G (18-24 million U per day for patients with normal renal function, divided into doses given every 4 h), can be a satisfactory alternative (Wormser G P, Clinical Infectious Diseases 2006; 43:1089-1134).

Patients with atrioventricular heart block and/or myopericarditis associated with early Lyme disease can be treated with either oral or parenteral antibiotic therapy for 14-21 days (Wormser G P, Clinical Infectious Diseases 2006; 43:1089-1134). Hospitalization and continuous monitoring are advisable for symptomatic patients, such as those with syncope, dyspnea, or chest pain. A parenteral antibiotic, such as ceftriaxone, is recommended as initial treatment of hospitalized patients (Wormser G P, Clinical Infectious Diseases 2006; 43:1089-1134). An oral antibiotic treatment regimen should be used for completion of therapy and for outpatients, as is used for patients with erythema migrans without carditis.

Lyme arthritis can usually be treated successfully with antimicrobial agents administered orally. Treatment with doxycycline (100 mg twice per day), amoxicillin (500 mg 3 times per day), or cefuroxime axetil (500 mg twice per day) for 28 days is recommended for adult patients without clinical evidence of neurologic disease. If patients treated with oral agents have subsequently manifested overt neuroborreliosis, intravenous administration of a β-lactam antibiotic may be required (Wormser G P, Clinical Infectious Diseases 2006; 43:1089-1134).

For patients who have persistent or recurrent joint swelling after a recommended course of oral antibiotic therapy, re-treatment with another 4-week course of oral antibiotics or with a 2-4-week course of ceftriaxone IV after several months is recommended by the CDC (Wormser G P, Clinical Infectious Diseases 2006; 43:1089-1134). Intravenous antibiotic therapy can be used for those patients whose arthritis failed to improve at all or worsened. If patients have no resolution of arthritis despite intravenous therapy, symptomatic treatment by nonsteroidal anti-inflammatory agents, intra-articular injections of corticosteroids, or disease-modifying antirheumatic drugs (DMARDs), such as hydroxychloroquine, can be used (Wormser G P, Clinical Infectious Diseases 2006; 43:1089-1134).

Adult patients with late neurologic disease affecting the central or peripheral nervous system can be treated with intravenous ceftriaxone for 2 to 4 weeks, with intravenous cefotaxime or penicillin G as alternatives (Wormser G P, Clinical Infectious Diseases 2006; 43:1089-1134).

In some embodiments, the methods provided herein also include treatment of a co-infection of Babesia, Bartonella, Ehrlichia, Anaplasma, and/or Rickettsia. Standard therapeutic regimens for Babesia can include administration of medicaments such as atovaquone (Mepron) plus azithromycin (Zithromax), clindamycin and oral quinine Standard therapeutic regimens for Bartonella can include administration of medicaments such as erythromycin, fluoroquinolone, or rifampin. Ehrlichia is frequently treated with the administration of medicaments such as doxycycline and rifampin.

### Compositions Comprising One or More Borrelia Antigens

Borrelia antigens are polypeptides or proteins having one or more immunoreactive epitopes, which are derived from or exhibiting sequence similarity to polypeptides or proteins derived from one or more pathogenic species of Borrelia: e.g., Borrelia burgdorferi sensu stricto, Borrelia afzelii, Borrelia garinii, Borrelia valaisiana, Borrelia bissettii, Borrelia lusitaniae, and Borrelia spielmanii (Chu et al., Journal of Medical Microbiology 57: 980-985, 2008). The terms “polypeptide” and “protein” are used interchangeably herein and refer to any peptide-linked chain of amino acids, regardless of length or post-translational modification. A polypeptide for use in the materials and methods described herein can be an antigenic fragment of any of the polypeptides described herein, provided the antigenic fragment includes at least one epitope of the reference polypeptide.

By way of example and without limitation, a polypeptide can be a native, recombinant, or chemically synthesized polypeptide or antigenic fragment thereof. In some embodiments, a polypeptide can be a substantially purified polypeptide obtained from a whole organism lysate. A substantially purified polypeptide or protein is substantially free of cellular material or other contaminating proteins from the cell or tissue source from which the protein is derived, or substantially free from chemical precursors or other chemicals if chemically synthesized. Any appropriate method for obtaining substantially pure polypeptides can be used.

In some embodiments, a polypeptide can be obtained by expression of a recombinant nucleic acid encoding the polypeptide or by chemical synthesis (e.g., by solid-phase synthesis or other methods well known in the art, including synthesis with an ABI peptide synthesizer; Applied Biosystems, Foster City, Calif.). Expression vectors that encode the polypeptide of interest can be used to produce a polypeptide. For example, standard recombinant technology using expression vectors encoding a polypeptide can be used. Expression systems that can be used for small or large-scale production of the polypeptides provided herein include, without limitation, microorganisms such as bacteria transformed with recombinant bacteriophage DNA, plasmid DNA, or cosmid DNA expression vectors containing the nucleic acid molecules of the polypeptide of interest. The resulting polypeptides can be purified according to any appropriate protein purification method. In some embodiments, substantially pure polypeptides or antigenic fragments thereof can be purchased from a commercial supplier (e.g., Diarect, Freiburg, Germany).

Antigens appropriate for the compositions and methods provided herein can be recombinant or synthetic polypeptides exhibiting a percent sequence identity to the native polypeptides derived from one or more species of Borrelia. As used herein, the term “percent sequence identity” refers to the degree of identity between any given query sequence and a subject sequence. Percentage of “sequence identity” is determined by comparing two optimally aligned sequences over a comparison window, where the fragment of the amino acid sequence in the comparison window may comprise additions or deletions (e.g., gaps or overhangs) as compared to the reference sequence (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity. The output is the percent identity of the subject sequence with respect to the query sequence. It is noted that a query nucleotide or amino acid sequence that aligns with a subject sequence can result in many different lengths, with each length having its own percent identity. Optimal alignment of sequences for comparison may be conducted by the local homology algorithm of Smith and Waterman (1981) Add. APL. Math. 2:482; by the homology alignment algorithm of Needleman and Wunsch (1970) J. Mol. Biol. 48:443; by the search for similarity method of Pearson and Lipman (1988) Proc. Natl. Acad. Sci. (USA) 85: 2444; by computerized implementations of algorithms such as GAP, BESTFIT, BLAST, PASTA, and TFASTA (Accelrys, Inc., 10188 Telesis Court, Suite 100 San Diego, Calif. 92121); or by inspection. Typically, the default values of 5.00 for gap weight and 0.30 for gap weight length are used.

The following Borrelia antigens are provided to demonstrate the utility of the current testing platform. OspC is an outer surface protein expressed as the spirochete traverses to the mammalian host, whereas related outer surface polypeptides OspA and OspB are mainly expressed in the mid-gut of the tick. Amino acid sequences of OspC proteins from Borrelia burgdorferi, Borrelia valaisiana, Borrelia garinii, and Borrelia afzelii are set forth in SEQ ID NOs:1-4, respectively. A Borrelia antigen for inclusion in a composition described herein can include an amino acid sequence having at least 80% (e.g., 80%, 85%, 90%, 95%, 100%) sequence identity to a Borrelia OspC polypeptide, or to an antigenic fragment thereof. In some embodiments, a Borrelia antigen can include an amino acid sequence having at least 80% (e.g., 80%, 85%, 90%, 95%, 100%) identity to a polypeptide selected from SEQ ID NOs:1-4, or to an antigenic fragment thereof. In some embodiments, a Borrelia antigen has an amino acid sequence of SEQ ID NO:1, 2, 3 or 4.

Amino acid sequences of OspA proteins from Borrelia burgdorferi, Borrelia valaisiana, Borrelia garinii, and Borrelia afzelii are set forth in SEQ ID NOs:16-18, respectively. A Borrelia antigen for inclusion in a composition described herein can include an amino acid sequence having at least 80% (e.g., 80%, 85%, 90%, 95%, 100%) sequence identity to a Borrelia OspA polypeptide, or to an antigenic fragment thereof. In some embodiments, a Borrelia antigen can include an amino acid sequence having at least 80% (e.g., 80%, 85%, 90%, 95%, 100%) identity to a polypeptide selected from SEQ ID NOs:16-18, or to an antigenic fragment thereof. In some embodiments, a Borrelia antigen has an amino acid sequence of SEQ ID NO:16, 17, or 18.

P100 is a high molecular weight major antigen of the membranous vesicle on the surface of Borrelia burgdorferi and is expressed late in Borrelia infection. Antibodies against P100 are usually of the IgG type and generally only appear in the chronic stage of the infection. Amino acid sequences of P100 protein from Borrelia burgdorferi is set forth in SEQ ID NO:5. A Borrelia antigen for inclusion in a composition described herein can include an amino acid sequence having at least 80% (e.g., 80%, 85%, 90%, 95%, 100%) sequence identity to a Borrelia P100 polypeptide, or to an antigenic fragment thereof. In some embodiments, a Borrelia antigen can include an amino acid sequence having at least 80% (e.g., 80%, 85%, 90%, 95%, 100%) identity to polypeptide SEQ ID NO:5, or to an antigenic fragment thereof. In some embodiments, a Borrelia antigen has an amino acid sequence of SEQ ID NO:5, or an antigenic fragment thereof.

Variable major protein-like E (VlsE) is an outer surface lipoprotein that undergoes antigenic variation during disseminated infection. The Borrelia bacterium is hidden from the immune system by antigenic variation of surface proteins expressed by VlsE genes. Thus, antibodies to VlsE can serve as a diagnostic marker of later stages of Borrelia infection. Amino acid sequences of VlsE proteins from Borrelia burgdorferi and Borrelia garinii are set forth in SEQ ID NOs:6-7, respectively. A Borrelia antigen for inclusion in a composition described herein can include an amino acid sequence having at least 80% (e.g., 80%, 85%, 90%, 95%, 100%) sequence identity to a Borrelia VlsE polypeptide, or to an antigenic fragment thereof. In some embodiments, a Borrelia antigen can include an amino acid sequence having at least 80% (e.g., 80%, 85%, 90%, 95%, 100%) identity to a polypeptide selected from SEQ ID NOs:6-7, or to an antigenic fragment thereof. In some embodiments, a Borrelia antigen has an amino acid sequence of SEQ ID NO: 6 or 7.

P41, or flagellin, is expressed in early and late Borrelia infection. Amino acid sequences of P41/flagellin proteins from Borrelia afzelii and Borrelia burgdorferi are set forth in SEQ ID NOs: 19-20. A Borrelia antigen for inclusion in a composition described herein can include an amino acid sequence having at least 80% (e.g., 80%, 85%, 90%, 95%, 100%) sequence identity to a Borrelia P41/flagellin polypeptide, or to an antigenic fragment thereof. In some embodiments, a Borrelia antigen can include an amino acid sequence having at least 80% (e.g., 80%, 85%, 90%, 95%, 100%) identity to a polypeptide selected from SEQ ID NOs:19-20, or to an antigenic fragment thereof. In some embodiments, a Borrelia antigen has an amino acid sequence of SEQ ID NO:19 or 20.

Additional antigens appropriate for the compositions provided herein are bacterial antigens that bind host proteins. For example, BmpA is a Borrelia membrane protein that enhances spirochete colonization and survival in host tissues. BmpA and its three paralogous proteins, BmpB, BmpC, and BmpD, bind mammalian laminin. Accordingly, polypeptides suitable for the compositions provided herein can have an amino acid sequence with at least 80% (e.g., 80%, 85%, 90%, 95%, 100%) sequence identity to a Borrelia BmpA, BmpB, BmpC, or BmpD protein. In some embodiments, a Borrelia antigen for inclusion in a composition described herein can include an amino acid sequence having at least 80% (e.g., 80%, 85%, 90%, 95%, 100%) sequence identity to BmpA amino acid sequences set forth as SEQ ID NO:21 (Borrelia burgdorferi), SEQ ID NO:22 (Borrelia garinii), or SEQ ID NO:23 (Borrelia afzelii). In some embodiments, a Borrelia antigen has an amino acid sequence of SEQ ID NO:21, 22, or 23.

Decorin-binding proteins A and B (DbpA and DbpB) bind decorin, a proteoglycan that associates with collagen. The decorin binding proteins promote binding of the spirochete to extracellular matrix proteins of host cells for maximum colonization of host tissues including skin and joints. Accordingly, polypeptides suitable for the compositions provided herein can have an amino acid sequence with at least 80% (e.g., 80%, 85%, 90%, 95%, 100%) sequence identity to a Borrelia DbpA. In some embodiments, a Borrelia antigen for inclusion in a composition described herein can include an amino acid sequence having at least 80% (e.g., 80%, 85%, 90%, 95%, 100%) sequence identity to DbpA amino acid sequences set forth as SEQ ID NO:8 (Borrelia burgdorferi), SEQ ID NO:9 (Borrelia garinii), or SEQ ID NO:10 (Borrelia afzelii). In some embodiments, a Borrelia antigen for inclusion in a composition described herein can include an amino acid sequence having at least 80% (e.g., 80%, 85%, 90%, 95%, 100%) sequence identity to DbpB amino acid sequences set forth as SEQ ID NO:11 (Borrelia burgdorferi), SEQ ID NO:12 (Borrelia garinii), or SEQ ID NO:13 (Borrelia afzelii). In some embodiments, a Borrelia antigen has an amino acid sequence of SEQ ID NO:11, 12, or 14.

Neutrophil activating protein (NapA) is a member of the Dps-like protein family with specific immunomodulatory properties. In particular, NapA can induce the expression of IL-23 in neutrophils and monocytes, as well as the expression of IL-6, IL-1β, and transforming growth factor β (TGF-β) in monocytes, via Toll-like receptor 2 (TLR2). NapA is the main Borrelia product involved in the pathogenesis of Lyme arthritis through accumulating and orchestrating the recruitment of inflammatory cells into the joint cavity. Accordingly, polypeptides suitable for the compositions provided herein can have an amino acid sequence with at least 80% (e.g., 80%, 85%, 90%, 95%, 100%) sequence identity to a Borrelia NapA. In some embodiments, a Borrelia antigen for inclusion in a composition described herein can include an amino acid sequence having at least 80% (e.g., 80%, 85%, 90%, 95%, 100%) sequence identity to NapA amino acid sequences set forth as SEQ ID NO:14 (Borrelia burgdorferi), or SEQ ID NO:15 (Borrelia afzelii). In some embodiments, a Borrelia antigen has an amino acid sequence of SEQ ID NO:14 or 15.

Other host receptor binding proteins can include P66, a 66-kilodalton (kD) spirochetal polypeptide that binds platelet-specific integrin α2bβ3 and the vitronectin receptor αvβ3; Bgp, a 26-kD glycosaminoglycan-binding polypeptide that binds heparin sulfate and dermatin sulphate; and Fbp, a 47 kD fibronectin-binding polypeptide. Accordingly, polypeptides exhibiting at least 80% (e.g., 80%, 85%, 90%, 95%, 100%) sequence identity to Borrelia P66, Bgp, or Fbp polypeptides are also suitable for inclusion.

Compositions provided herein typically include one or more (e.g., 1, 2, 3, 4, 5, 6, or more) Borrelia antigens selected from the group consisting of but not limited to a VlsE polypeptide or an antigenic fragment thereof, a NapA polypeptide or an antigenic fragment thereof, a DbpA polypeptide or an antigenic fragment thereof, a DbpB polypeptide or an antigenic fragment thereof, an OspC polypeptide or an antigenic fragment thereof, an OspA polypeptide or an antigenic fragment thereof, an OspB polypeptide or an antigenic fragment thereof, a P100 polypeptide or an antigenic fragment thereof, a P41 polypeptide or an antigenic fragment thereof, a P66 polypeptide or an antigenic fragment thereof, a BmpA polypeptide or an antigenic fragment thereof, a BmpB polypeptide or an antigenic fragment thereof, a BmpC polypeptide or an antigenic fragment thereof, a Bgp polypeptide or an antigenic fragment thereof, and a Fbp polypeptide or an antigenic fragment thereof. The polypeptides or antigenic fragment thereof can be native, recombinant, or chemically synthesized.

In some embodiments, the compositions provided herein can include a mixture of an OspC polypeptide or an antigenic fragment thereof and a VlsE polypeptide or an antigenic fragment thereof. In some embodiments, the compositions provided herein can include a mixture of a DbpA polypeptide or an antigenic fragment thereof, an OspC polypeptide or an antigenic fragment thereof, a P100 polypeptide or an antigenic fragment thereof, and a VlsE polypeptide or an antigenic fragment thereof.

In some embodiments, the compositions provided herein can include a mixture of a NapA polypeptide or an antigenic fragment thereof, with one or more polypeptides or antigenic fragments thereof selected from VlsE, DbpA, DbpB, OspC, OspA, OspB, P100, P41, P66, BmpA, BmpB, BmpC, Bgp, and Fbp. In some embodiments, the compositions provided herein can include a mixture of a NapA polypeptide or an antigenic fragment thereof, an OspC polypeptide or an antigenic fragment thereof and a VlsE polypeptide or an antigenic fragment thereof. In some embodiments, the compositions provided herein can include a mixture of a NapA polypeptide or an antigenic fragment thereof, a DbpA polypeptide or an antigenic fragment thereof, an OspC polypeptide or an antigenic fragment thereof, a P100 polypeptide or an antigenic fragment thereof, and a VlsE polypeptide or an antigenic fragment thereof.

In some embodiments, the compositions provided herein can include a mixture of an OspA polypeptide or an antigenic fragment thereof, with one or more polypeptides or antigenic fragments thereof selected from NapA, VlsE, DbpA, DbpB, OspC, OspB, P100, P41, P66, BmpA, BmpB, BmpC, Bgp, and Fbp. In some embodiments, the compositions provided herein can include a mixture of an OspA polypeptide or an antigenic fragment thereof, an OspC polypeptide or an antigenic fragment thereof and a VlsE polypeptide or an antigenic fragment thereof. In some embodiments, the compositions provided herein can include a mixture of an OspA polypeptide or an antigenic fragment thereof, a DbpA polypeptide or an antigenic fragment thereof, an OspC polypeptide or an antigenic fragment thereof, a P100 polypeptide or an antigenic fragment thereof, and a VlsE polypeptide or an antigenic fragment thereof.

In some embodiments, the compositions provided herein can include a mixture of a NapA polypeptide or an antigenic fragment thereof, an OspA polypeptide or an antigenic fragment thereof, an OspC polypeptide or an antigenic fragment thereof, and a Vlse polypeptide or an antigenic fragment thereof.

In some embodiments, the compositions provided herein can include a mixture of a NapA polypeptide or an antigenic fragment thereof, an OspA polypeptide or an antigenic fragment thereof, a DbpA polypeptide or an antigenic fragment thereof, an OspC polypeptide or an antigenic fragment thereof, a P100 polypeptide or an antigenic fragment thereof, and a Vlse polypeptide or an antigenic fragment thereof.

In some embodiments, the compositions provided herein can include an OspC polypeptide comprising an amino acid sequence having at least 80% (e.g., 80%, 85%, 90%, 95%, 100%) sequence identity to any one of SEQ ID NOs:1-4, or to an antigenic fragment thereof. In some embodiments, the compositions provided herein can include an OspC polypeptide having an amino acid sequence of any one of SEQ ID NOs:1-4. In some embodiments, the compositions provided herein can include an OspC polypeptide having an amino acid sequence of SEQ ID NO:1.

In some embodiments, the compositions provided herein can include a P100 polypeptide comprising an amino acid sequence having at least 80% (e.g., 80%, 85%, 90%, 95%, 100%) sequence identity to SEQ ID NOs:5, or to an antigenic fragment thereof. In some embodiments, the compositions provided herein can include an P100 polypeptide having an amino acid sequence of SEQ ID NOs:5.

In some embodiments, the compositions provided herein can include a VlsE polypeptide comprising an amino acid sequence having at least 80% (e.g., 80%, 85%, 90%, 95%, 100%) sequence identity to any one of SEQ ID NOs:6-7, or to an antigenic fragment thereof. In some embodiments, the compositions provided herein can include a VlsE polypeptide having an amino acid sequence of any one of SEQ ID NOs:6-7. In some embodiments, the compositions provided herein can include a VlsE polypeptide having an amino acid sequence of SEQ ID NO:6.

In some embodiments, the compositions provided herein can include a DbpA polypeptide comprising an amino acid sequence having at least 80% (e.g., 80%, 85%, 90%, 95%, 100%) sequence identity to any one of SEQ ID NOs:8-10, or to an antigenic fragment thereof. In some embodiments, the compositions provided herein can include a DbpA polypeptide having an amino acid sequence of any one of SEQ ID NOs:8-10. In some embodiments, the compositions provided herein can include a DbpA polypeptide having an amino acid sequence of SEQ ID NO:8.

In some embodiments, the compositions provided herein can include a DbpB polypeptide comprising an amino acid sequence having at least 80% (e.g., 80%, 85%, 90%, 95%, 100%) sequence identity to any one of SEQ ID NOs:11-13, or to an antigenic fragment thereof. In some embodiments, the compositions provided herein can include a DbpB polypeptide having an amino acid sequence of any one of SEQ ID NOs: 11-13. In some embodiments, the compositions provided herein can include a DbpB polypeptide having an amino acid sequence of SEQ ID NO:11.

In some embodiments, the compositions provided herein can include a NapA polypeptide comprising an amino acid sequence having at least 80% (e.g., 80%, 85%, 90%, 95%, 100%) sequence identity to any one of SEQ ID NOs:14-15, or to an antigenic fragment thereof. In some embodiments, the compositions provided herein can include a NapA polypeptide having an amino acid sequence of any one of SEQ ID NOs:14-15. In some embodiments, the compositions provided herein can include a NapA polypeptide having an amino acid sequence of SEQ ID NO:14.

In some embodiments, the compositions provided herein can include an OspA polypeptide comprising an amino acid sequence having at least 80% (e.g., 80%, 85%, 90%, 95%, 100%) sequence identity to any one of SEQ ID NOs:16-18, or to an antigenic fragment thereof. In some embodiments, the compositions provided herein can include a NapA polypeptide having an amino acid sequence of any one of SEQ ID NOs:16-18. In some embodiments, the compositions provided herein can include a NapA polypeptide having an amino acid sequence of SEQ ID NO:16.

In some embodiments, the compositions provided herein can include a P41 polypeptide comprising an amino acid sequence having at least 80% (e.g., 80%, 85%, 90%, 95%, 100%) sequence identity to any one of SEQ ID NOs:19-21, or to an antigenic fragment thereof. In some embodiments, the compositions provided herein can include a P41 polypeptide having an amino acid sequence of any one of SEQ ID NOs:19-20. In some embodiments, the compositions provided herein can include a P41 polypeptide having an amino acid sequence of SEQ ID NO:19.

In some embodiments, the compositions provided herein can include a BmpA polypeptide comprising an amino acid sequence having at least 80% (e.g., 80%, 85%, 90%, 95%, 100%) sequence identity to any one of SEQ ID NOs:21-23, or to an antigenic fragment thereof. In some embodiments, the compositions provided herein can include a BmpA polypeptide having an amino acid sequence of any one of SEQ ID NOs:21-23. In some embodiments, the compositions provided herein can include a BmpA polypeptide having an amino acid sequence of SEQ ID NO:21.

Concurrent infections of Lyme disease and other tick-borne illnesses can occur. Thus, a composition provided herein can include one or more antigens derived from or exhibiting sequence similarity to one or more tick-borne infectious agents. For example, a composition can include one or more polypeptides derived from a species of the protozoan parasite Babesia (e.g., Babesia bovis, Babesia divergens, Babesia microti). A composition can include one or more polypeptides derived from a species of the Gram-negative bacterium Bartonella (e.g., Bartonella bacilliformis, Bartonella henselae, Bartonella quintana, Bartonella rochalimae), from a species of the rickettsiales bacteria genus Anaplasma (e.g., Anaplasma phagocytophilum) or the genus Ehrlichia (e.g., Ehrlichia ewingii, Ehrlichia chaffeenis, Ehrlichia canis, Neorickettsia sennetsu), from a species of mycoplasma bacteria (e.g., Mycoplasma fermentans, Mycoplasma hominis, Mycoplasma pneumoniae, Mycoplasma genitalium, Mycoplasma penetrans), or from a species of Rickettsia (e.g., Rickettsia rickettsii, Rickettsia typhi) and others.

### Kits

Also provided herein are kits for detecting active Borrelia infection. These kits can include (1) a solid phase support, e.g., a particle or a microwell of a microplate, which is coated with one or more capture antibodies specific for one or more proteins secreted by Lyme antigen-specific CD8 effector T cells, e.g., antibodies that bind granzyme B, perforin, or granulysin, and (2) a composition comprising one or more Borrelia antigen polypeptides or antigenic fragments thereof as described herein. For example, the composition comprising one or more Borrelia antigen polypeptides can include one or more polypeptides of NapA, VlsE, DbpA, DbpB, OspC, OspA, OspB, P100, P41, P66, BmpA, BmpB, BmpC, Bgp, and Fbp. The kit can include one or more other elements including: instructions for use and other reagents such as serum-free medium; IL-7; a positive control, e.g., phytohaemagglutinin (PHA); a detection antibody for the one or more proteins secreted by Lyme antigen-specific CD8 effector T cells, e.g., antibodies that bind granzyme B, perforin, or granulysin; a substrate; buffers and antimicrobial agents. The capture and detection antibodies can be monoclonal or polyclonal antibodies that bind to different epitopes on the proteins secreted by Lyme antigen-specific CD8 effector T cells. The detection antibody can be any detectably labeled antibody, for example, an antibody tagged with a fluorescent dye, e.g., an Alexa Fluor 488-conjugated antibody; an enzyme-conjugated antibody, e.g., alkaline phosphatase-conjugated antibody; or an antibody conjugated with one member of a specific binding pair, e.g., an antibody conjugated with biotin or streptavidin. For example, if a biotinylated detection antibody is included in the kit, the kit also includes enzyme-conjugated streptavidin, e.g., alkaline phosphatase-conjugated streptavidin. The kit can include a chromogenic, fluorogenic, or electrochemiluminescent substrate of the enzyme on the detection antibody or strepavidin. For example, a chromogenic substrate for alkaline phosphatase can be a 5-Bromo-4-chloro-3-indolyl phosphate (BCIP), nitro blue tetrazolium chloride (NBT), or a mixture of BCIP and NBT. The instructions for use can be in a paper format or on a CD or DVD.

Kits as provided herein can be used in accordance with any of the methods described above, e.g., detecting or treating active Borrelia infection. Those skilled in the art will be aware of other suitable uses for kits provided herein, and will be able to employ the kits for such uses. Kits as provided herein can also include a mailer (e.g., a postage paid envelope or mailing pack) that can be used to return the sample for analysis, e.g., to a laboratory. The kit can include one or more containers for the sample, or the sample can be in a standard blood collection vial. The kit can also include one or more of an informed consent form, a test requisition form, and instructions on how to use the kit in a method described herein. Methods for using such kits are also included herein. One or more of the forms (e.g., the test requisition form) and the container holding the sample can be coded, for example, with a bar code for identifying the subject who provided the sample.

### Examples

The invention is further described in the following examples, which do not limit the scope of the invention described in the claims.

### Example 1

### Granzyme B Lyme ELISpot Assay

The Granzyme B Lyme ELISpot assays were performed to measure the number of Borrelia antigen-specific CD8 effector T cells. Human peripheral blood mononuclear cells (PBMCs) were isolated from whole blood samples of diagnosed Lyme patients. All individuals that were classified as Lyme patients met the CDC surveillance definition of Lyme disease, including clinical signs and symptoms, history of possible exposure to infected blacklegged ticks, with or without a positive antibody response to Borrelia burgdorferi by ELISA and Western Blot, interpreted according to CDC and the Infectious Disease Society of America (IDSA) criteria.

The PBMCs were plated at 250,000 cells per well in anti-Granzyme B antibody pre-coated 96-well plates. The PBMCs were then stimulated with recombinant Borrelia antigens in serum-free medium, in the presence of Interleukin-7 (1.3 ng/ml, R&D Systems, MN, USA). Serum-free medium alone served as a negative control. All Borrelia antigens were purchased from DIARECT AG (Freiberg, Germany). All culture conditions (negative control, positive control, and Borrelia antigen stimulation) were tested in triplicate. PBMCs were incubated with the Borrelia antigen for about 18-24 hours at 37° C., 9% CO₂. During the incubation period, Granzyme B secreted by Borrelia antigen-specific CD8 effector T cells was captured by the anti-Granzyme B antibody immobilized on PVDF membranes. After incubation, the wells were washed with serum-free medium to remove any unbound material. A biotinylated anti-Granzyme B antibody directed against a different epitope on Granzyme B was added to the wells and allowed to react for 2 hours. The wells were washed again, and streptavidin conjugated alkaline-phosphatase (Strep-AP) were added to each well. After unbound Strep-AP was removed by washing, a mixture of 5-Bromo-4-chloro-3-indolyl phosphate and nitro blue tetrazolium chloride was added to the wells. A blue-colored precipitate formed and appeared as a spot at each Granzyme B-antibody complex. The wells are washed again and dried overnight prior to plate analysis. The results of ELISPOT were analyzed using the CTL S6 Ultimate-V Analyzer/BioSpot 5.0 Software (CTL, OH, USA) and reported as Granzyme B Spot Forming Units (SFU). Parameters for the analyzer were set to be: spot separation=1, diffuse processing=Large, adjusted count area=95%, fiber removal=yes. The number of spot forming units (SFU) was counted by an automated ImmunoSpot reader. The number of spots represents the number of Borrelia antigen-specific CD8 effector T cells.

In some assays, the Borrelia antigen used was a recombinant DbpA polypeptide having an amino acid sequences of SEQ ID NO:8. In some assays, the Borrelia antigen used was a recombinant OspC polypeptide having an amino acid sequences of SEQ ID NO:1. In some assays, the Borrelia antigen used was a recombinant P100 polypeptide having an amino acid sequences of SEQ ID NO:5. In some assays, the Borrelia antigen used was a recombinant VlsE polypeptide having an amino acid sequences of SEQ ID NO:6. In some assays, the Borrelia antigen used was a recombinant NapA polypeptide having an amino acid sequences of SEQ ID NO:14. In some assays, the Borrelia antigens used was Antigen mix 1, a mixture of a recombinant OspC polypeptide having an amino acid sequences of SEQ ID NO:1, a recombinant P100 polypeptide having an amino acid sequences of SEQ ID NO:5, a recombinant VlsE polypeptide having an amino acid sequences of SEQ ID NO:6, and a recombinant DbpA polypeptide having an amino acid sequences of SEQ ID NO:8. In some assays, the Borrelia antigen used was Antigen mix 3, a mixture of a recombinant OspC polypeptide having an amino acid sequences of SEQ ID NO:1, a recombinant P100 polypeptide having an amino acid sequences of SEQ ID NO:5, a recombinant VlsE polypeptide having an amino acid sequences of SEQ ID NO:6, a recombinant DbpA polypeptide having an amino acid sequences of SEQ ID NO:8, and a recombinant NapA polypeptide having an amino acid sequences of SEQ ID NO:14.

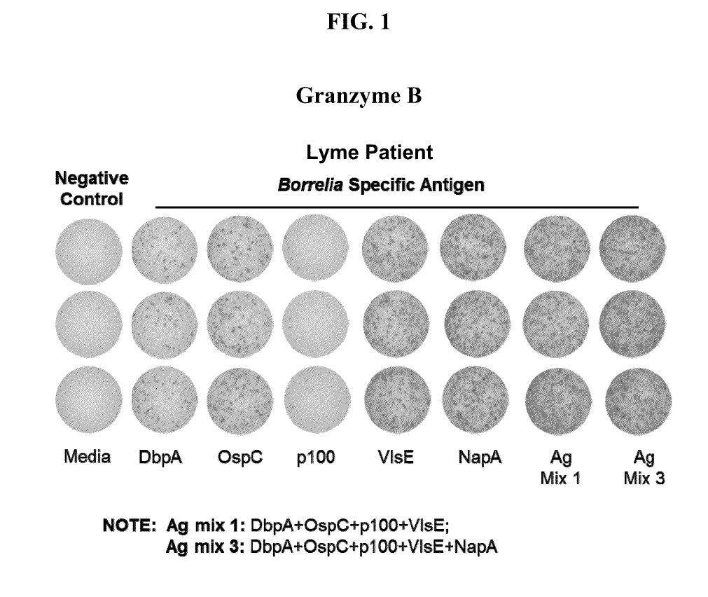

FIG. 1 shows representative Granzyme B Lyme ELISpot results for a Lyme patient. The Borrelia antigens used were: DbpA alone, OspC alone, P100 alone, VlsE alone, NapA alone, Antigen mix 1 or Antigen mix 3. Serum-free medium alone serves as a negative control. All Borrelia antigens tested stimulated Granzyme B secretion by the Borrelia antigen-specific CD8 effector T cells while no Granzyme B secretion was detected in the media controls (FIG. 1), indicating the Lyme patient has active Borrelia infection.

### Example 2

### IL-17 Lyme ELISpot Assay

The IL-17 Lyme ELISpot assays were performed to measure the number of Borrelia antigen-specific Th17 cells. Peripheral blood mononuclear cells (PBMCs) were isolated from whole blood samples of diagnosed Lyme patients, and plated at 250,000 cells per well in anti-IL-17 antibody pre-coated 96-well plates. The PBMCs were then stimulated with recombinant Borrelia antigens in serum-free medium, in the presence of Interleukin-7 (1.3 ng/ml, R&D Systems, MN, USA). Serum-free medium alone served as a negative control; phytohaemagglutinin (PHA) served as a positive control. All Borrelia antigens were purchased from DIARECT AG (Freiberg, Germany). All culture conditions (negative control, positive control, and Borrelia antigen stimulation) were tested in triplicate. PBMCs were incubated with Borrelia antigen for about 18-24 hours at 37° C., 9% CO₂. During the incubation period, IL-17 secreted by Borrelia antigen-specific Th17 cells was captured by the anti-IL-17 antibody immobilized on PVDF membranes. After incubation, the wells were washed with serum-free medium to remove any unbound material. A biotinylated anti-IL-17 antibody directed against a different epitope on IL-17 was added to the wells and allowed to react for 2 hours. The wells were washed again, and streptavidin conjugated alkaline-phosphatase (Strep-AP) were added to each well. After unbound Strep-AP was removed by washing, a mixture of 5-Bromo-4-chloro-3-indolyl phosphate and nitro blue tetrazolium chloride was added to the wells. A blue-colored precipitate formed and appeared as a spot at each IL-17-antibody complex. The wells are washed again and dried overnight prior to plate analysis. The results of ELISPOT were analyzed using the CTL S6 Ultimate-V Analyzer/BioSpot 5.0 Software (CTL, OH, USA) and reported as IL17 Spot Forming Units (SFU). Parameters for the analyzer were set to be: spot separation=1, diffuse processing=Large, adjusted count area=95%, fiber removal=yes. The number of spot forming units (SFU) was counted by an automated ImmunoSpot reader. The number of spots represents the number of Borrelia antigen-specific Th17 cells.

In some assays, the Borrelia antigen used was a recombinant NapA polypeptide having an amino acid sequences of SEQ ID NO:14. In some assays, the Borrelia antigens used was Antigen mix 1, a mixture of a recombinant OspC polypeptide having an amino acid sequences of SEQ ID NO:1, a recombinant P100 polypeptide having an amino acid sequences of SEQ ID NO:5, a recombinant VlsE polypeptide having an amino acid sequences of SEQ ID NO:6, and a recombinant DbpA polypeptide having an amino acid sequences of SEQ ID NO:8. In some assays, the Borrelia antigen used was Antigen mix 3, a mixture of a recombinant OspC polypeptide having an amino acid sequences of SEQ ID NO:1, a recombinant P100 polypeptide having an amino acid sequences of SEQ ID NO:5, a recombinant VlsE polypeptide having an amino acid sequences of SEQ ID NO:6, a recombinant DbpA polypeptide having an amino acid sequences of SEQ ID NO:8, and a recombinant NapA polypeptide having an amino acid sequences of SEQ ID NO:14.

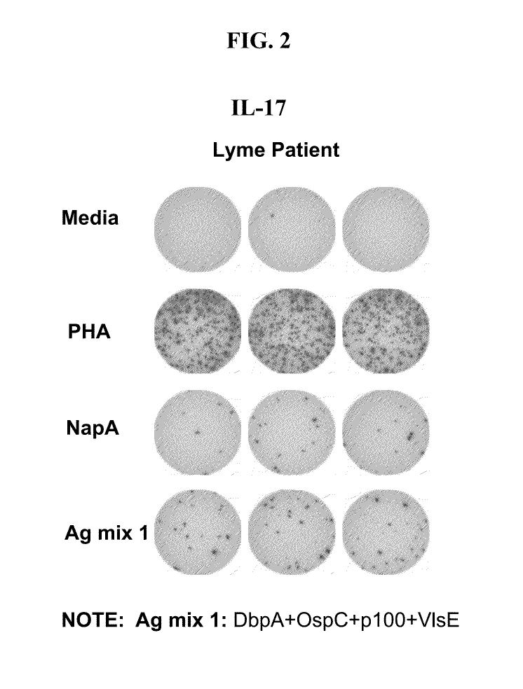

FIG. 2 shows representative IL-17 Lyme ELISpot results for a Lyme patient. NapA alone or Antigen mix 1 was used to stimulate the PBMCs. Serum-free medium alone serves as a negative control; PHA served as a positive control. Both NapA alone and Antigen mix 1 stimulated IL-17 secretion by the Borrelia antigen-specific Th17 cells while no IL-17 secretion was detected in the media controls (FIG. 2), confirming that the IL-17 Lyme ELISpot assay is able to detect Borrelia infection in Lyme patient.

### Example 3

### IFN-γ Lyme ELISpot Assay

The IFN-γ Lyme ELISpot assays were performed to measure the total number of Borrelia antigen-specific T cells. Human peripheral blood mononuclear cells (PBMCs) were isolated from whole blood samples, obtained from a cohort of healthy donors and diagnosed Lyme patients. The PBMCs were plated at 250,000 cells per well in anti-IFN-γ antibody pre-coated 96-well plates (part of human IFN-γ ELISPOT kit by Cellular Technology Limited, OH, USA). The PBMCs were then stimulated with recombinant Borrelia antigens in serum-free medium, in the presence or absence of Interleukin-7 (1.3 ng/ml, R&D Systems, MN, USA). Serum-free medium alone served as a negative control, and phytohaemagglutinin (PHA), a known activator of T-lymphocytes, was used as positive control. All Borrelia antigens were purchased from DIARECT AG (Freiberg, Germany). All culture conditions (negative control, positive control, and Borrelia antigen stimulation) were tested in triplicate. PBMCs were incubated for 18-24 hours at 37° C., 9% CO₂. During the incubation period, IFN-γ secreted by Borrelia antigen-specific T-cells was captured by the anti-IFN-γ antibody immobilized on PVDF membranes. After incubation, the wells were washed with serum-free medium to remove any unbound material. A biotinylated anti-IFN-γ antibody directed against a different epitope on IFN-γ (part of human IFN-γ ELISPOT kit by Cellular Technology Limited, OH, USA), was added to the wells and allowed to react for 2 hours. The wells were washed again, and streptavidin conjugated alkaline-phosphatase (Strep-AP) were added to each well. After unbound Strep-AP was removed by washing, a mixture of 5-Bromo-4-chloro-3-indolyl phosphate and nitro blue tetrazolium chloride was added to the wells. A blue-colored precipitate formed and appeared as a spot at each IFN-γ-antibody complex. The wells are washed again and dried overnight prior to plate analysis. The results of ELISPOT were analyzed using the CTL S6 Ultimate-V Analyzer/BioSpot 5.0 Software (CTL, OH, USA) and reported as IFN-γ Spot Forming Units (SFU). Parameters for the analyzer were set to be: spot separation=1, diffuse processing=Large, adjusted count area=95%, fiber removal=yes. The number of spot forming units (SFU) was counted by an automated ImmunoSpot reader. The number of spots represents the number of Borrelia antigen-specific T cells.

In some assays, the Borrelia antigens used was Antigen mix 1, a mixture of a recombinant OspC polypeptide having an amino acid sequences of SEQ ID NO:1, a recombinant P100 polypeptide having an amino acid sequences of SEQ ID NO:5, a recombinant VlsE polypeptide having an amino acid sequences of SEQ ID NO:6, and a recombinant DbpA polypeptide having an amino acid sequences of SEQ ID NO:8. In some assays, the Borrelia antigens used was Antigen mix 2, a mixture of a recombinant OspC polypeptide having an amino acid sequences of SEQ ID NO:1 and a recombinant VlsE polypeptide having an amino acid sequences of SEQ ID NO:6. In some assays, the Borrelia antigen used was Antigen mix 3, a mixture of a recombinant OspC polypeptide having an amino acid sequences of SEQ ID NO:1, a recombinant P100 polypeptide having an amino acid sequences of SEQ ID NO:5, a recombinant VlsE polypeptide having an amino acid sequences of SEQ ID NO:6, a recombinant DbpA polypeptide having an amino acid sequences of SEQ ID NO:8, and a recombinant NapA polypeptide having an amino acid sequences of SEQ ID NO:14.

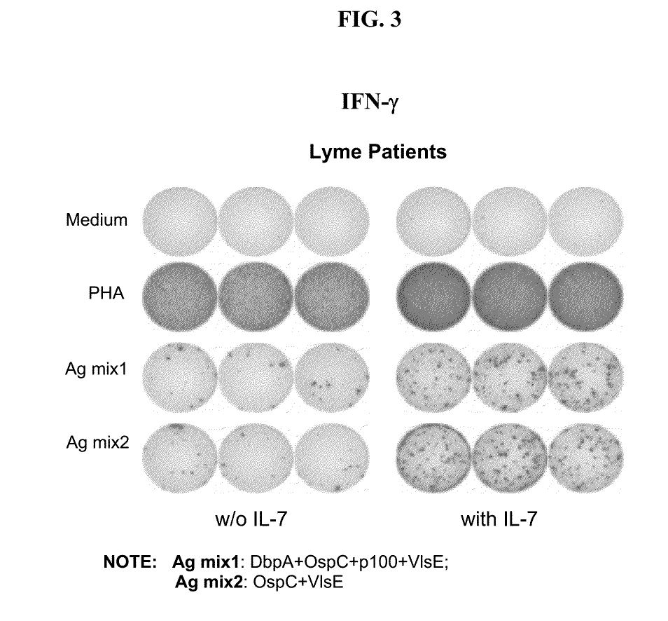

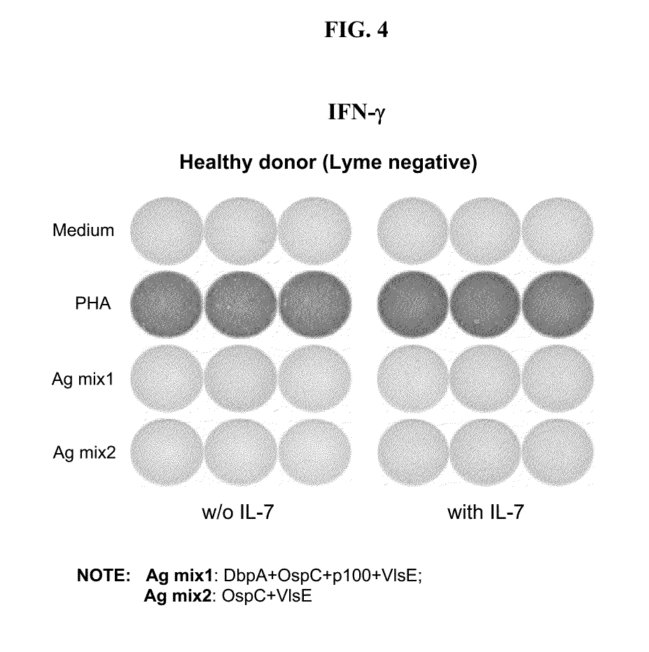

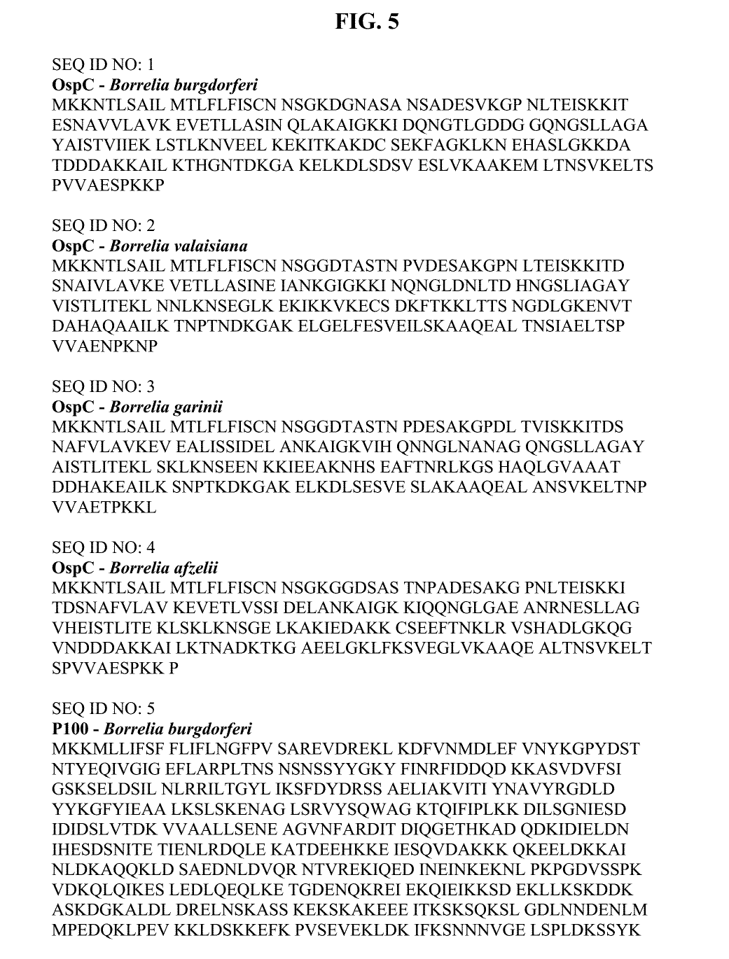

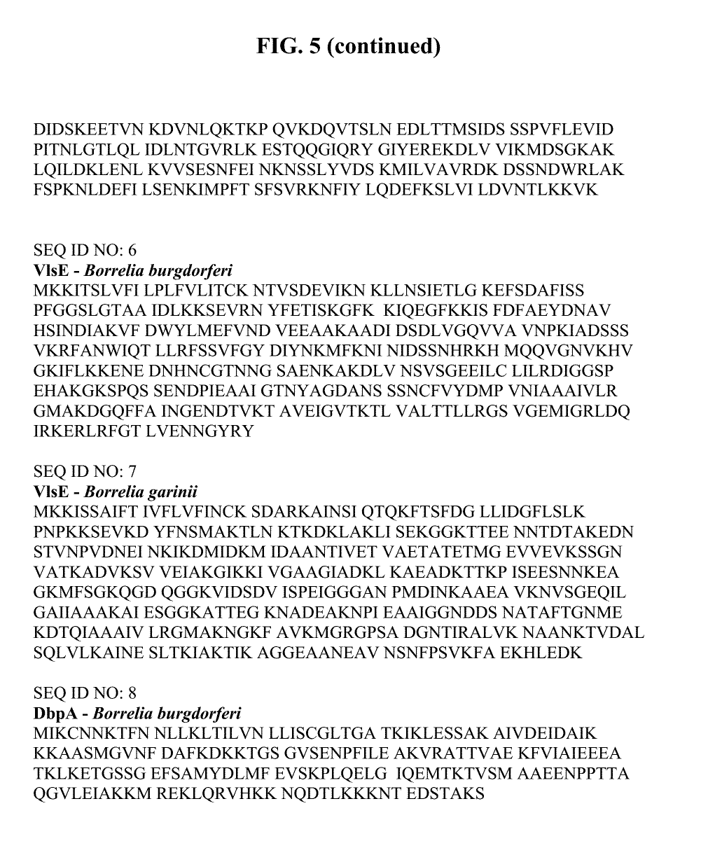

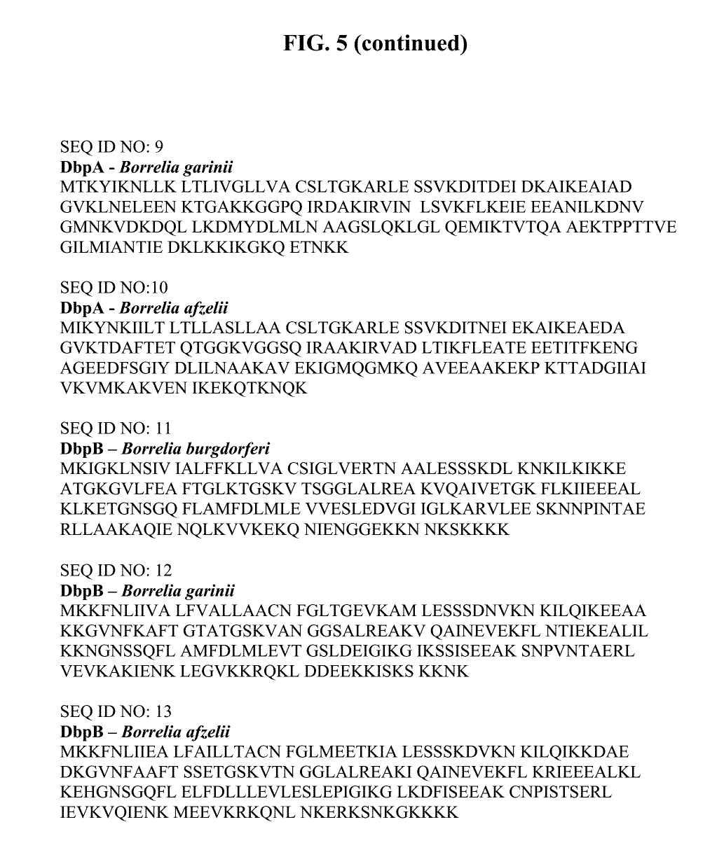

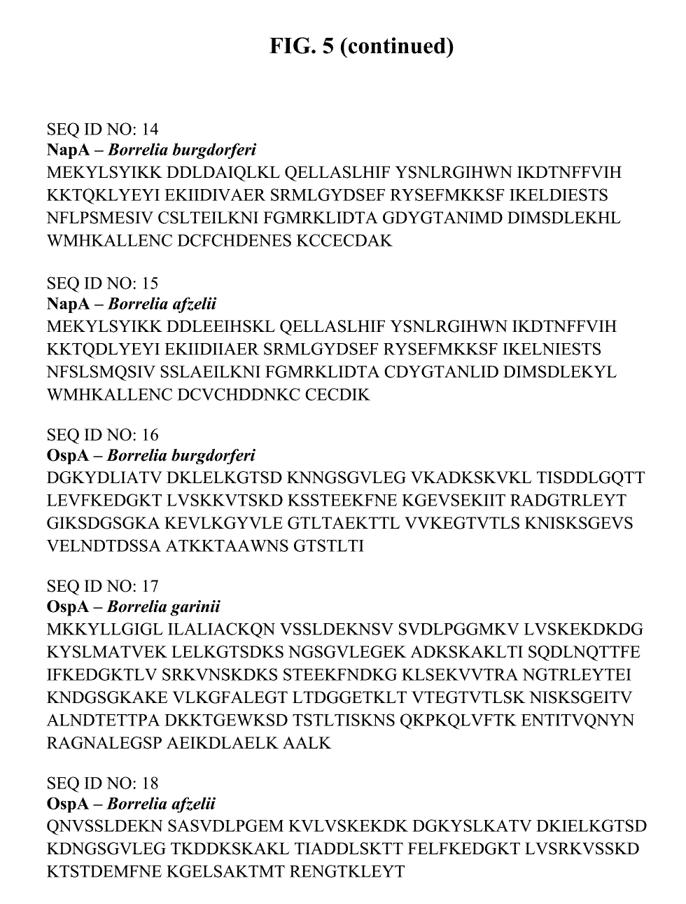

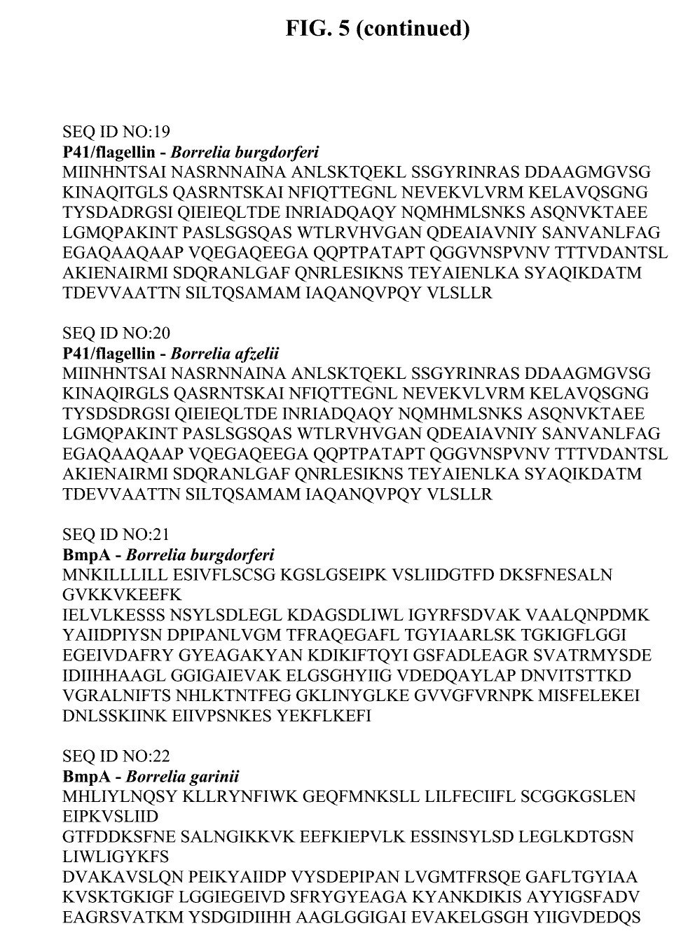

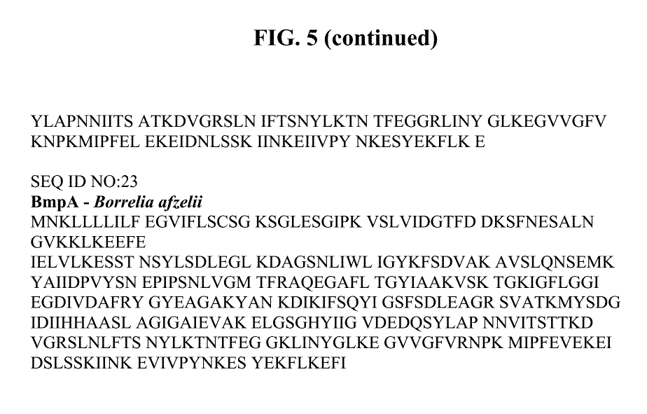

FIGS. 3-4 show representative IFN-γ Lyme ELISpot results for a Lyme patient (FIG. 3) and a healthy donor (FIG. 4). The Borrelia antigen mixtures used were: Antigen mix 1 and Antigen mix 2. Serum-free medium alone serves as a negative control, and PHA (phytohaemagglutinin) serves as a positive control. Both the IFN-γ Lyme ELISpot assay with IL-7 and the IFN-γ Lyme ELISpot assay without IL-7 reported positive results for the Lyme patient, and the addition of IL-7 dramatically amplified the signal of IFN-γ-forming spots (FIG. 3). Both the Lyme ELISpot assays (with or without IL-7) reported negative results for the healthy donor (FIG. 4). These findings indicated that the IFN-γ Lyme ELISpot assays can be used to detect Borrelia antigen-specific T cells. The addition of IL-7 improved the detection of these Borrelia antigen-specific T cells, without increasing non-specific spots in healthy controls and the medium control background.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for detecting active Borrelia infection in a subject, the method comprising:
a. providing peripheral blood mononuclear cells (PBMCs) of the subject;
b. incubating the PBMCs in a serum-free medium with one or more Borrelia antigens for about 18 to about 72 hours; and
c. determining the ratio of Borrelia antigen-specific CD8 effector T cells to PBMCs by measuring the level of one or more proteins secreted by Borrelia antigen-specific CD8 effector T cells, wherein the ratio of Borrelia antigen-specific CD8 effector T cells to PBMCs above a reference level is indicative of active Borrelia infection in the subject.

2. The method of claim 1, wherein the one or more proteins secreted by Borrelia antigen-specific CD8 effector T cells are selected from granzyme B, perforin, and granulysin.

3. The method of claim 1, wherein the reference level is the ratio of Borrelia antigen-specific CD8 effector T cells to PBMCs in a healthy subject.

4. The method of claim 1, further comprising measuring one or more cytokines secreted by the PBMCs, wherein the cytokines are selected from the group consisting of IL-17, IFN-γ, IL-2, IL-3, IL-4, IL-5, IL-6, IL-9, IL-10, IL-13, IL-21, IL-22, IL-25, IL-31, TNF-α, TNF-β, and GM-CSF.

5. The method of claim 4, further comprising one or more of the following steps:
d. determining the ratio of granzyme B-secreting CD8 T cells to PBMCs;
e. determining the ratio of IFN-γ-secreting CD8 T cells to PBMCs;
f. comparing the ratio of granzyme B-secreting CD8 T cells with the ratio of IFN-γ-secreting CD8 T cells; and
g. determining whether the subject has latent Borrelia infection.

6. (canceled)

7. (canceled)

8. (canceled)

9. The method of claim 1, wherein the one or more Borrelia antigens are polypeptides or proteins derived from or exhibiting sequence similarity to polypeptides or proteins derived from one or more pathogenic species of Borrelia.

10. (canceled)

11. The method of claim 9, wherein the one or more Borrelia antigens are selected from the group consisting of but not limited to: a Variable major protein-like gene E (VlsE) polypeptide or an antigenic fragment thereof, a Neutrophil activating protein (NapA) polypeptide or an antigenic fragment thereof, a Decorin-binding protein A (DbpA) polypeptide or an antigenic fragment thereof, a Decorin-binding protein B (DbpB) polypeptide or an antigenic fragment thereof, an Outer surface protein C (OspC) polypeptide or an antigenic fragment thereof, an Outer surface protein A (OspA) polypeptide or an antigenic fragment thereof, an Outer surface protein B (OspB) polypeptide or an antigenic fragment thereof, a P100 polypeptide or an antigenic fragment thereof, a P41 polypeptide or an antigenic fragment thereof, a P66 polypeptide or an antigenic fragment thereof, a Borrelia membrane protein A (BmpA) polypeptide or an antigenic fragment thereof, a Borrelia membrane protein B (BmpB) polypeptide or an antigenic fragment thereof, a Borrelia membrane protein C (BmpC) polypeptide or an antigenic fragment thereof, a Borrelia glycosaminoglycan-binding protein (Bgp) polypeptide or an antigenic fragment thereof, and a Fibronectin-binding protein (Fbp) polypeptide or an antigenic fragment thereof.

12. The method of claim 9, wherein the one or more Borrelia antigens are purified recombinant or synthetic polypeptides.

13. The method of claim 11, wherein the one or more Borrelia antigens are a mixture of a DbpA polypeptide or an antigenic fragment thereof, an OspC polypeptide or an antigenic fragment thereof, a P100 polypeptide or an antigenic fragment thereof, and a VlsE polypeptide or an antigenic fragment thereof.

14. The method of claim 11, wherein the one or more Borrelia antigens are a mixture of an OspC polypeptide or an antigenic fragment thereof and a VlsE polypeptide or an antigenic fragment thereof.

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. A method of treating active Borrelia infection in a subject, the method comprising:
a. providing peripheral blood mononuclear cells (PBMCs) of the subject;
b. incubating the PBMCs in a serum-free medium with one or more Borrelia antigens for about 18 to about 72 hours;
c. determining the ratio of Borrelia antigen-specific CD8 effector T cells to PBMCs by measuring the level of one or more proteins secreted by Borrelia antigen-specific CD8 effector T cells, and
d. if the ratio of Borrelia antigen-specific CD8 effector T cells to PBMCs is above a reference level, administering to the subject a treatment suitable for treating active Borrelia infection.

37. The method of claim 36, wherein the treatment suitable for treating active Borrelia infection comprises administering one or more antibiotics.

38. (canceled)

39. The method of claim 36, wherein the one or more proteins secreted by Borrelia antigen-specific CD8 effector T cells are selected from granzyme B, perforin, and granulysin.

40. (canceled)

41. The method of claim 36, further comprising measuring one or more cytokines secreted by the PBMCs, wherein the cytokines are selected from the group consisting of IL-17, IFN-γ, IL-2, IL-3, IL-4, IL-5, IL-6, IL-9, IL-10, IL-13, IL-21, IL-22, IL-25, IL-31, TNF-α, TNF-β, and GM-CSF.

42. (canceled)

43. (canceled)

44. (canceled)

45. The method of claim 36, wherein the one or more Borrelia antigens are polypeptides or proteins derived from or exhibiting sequence similarity to polypeptides or proteins derived from one or more pathogenic species of Borrelia.

46. (canceled)

47. The method of claim 45, wherein the one or more Borrelia antigens are selected from the group consisting of but not limited to: a Variable major protein-like gene E (VlsE) polypeptide or an antigenic fragment thereof, a Neutrophil activating protein (NapA) polypeptide or an antigenic fragment thereof, a Decorin-binding protein A (DbpA) polypeptide or an antigenic fragment thereof, a Decorin-binding protein B (DbpB) polypeptide or an antigenic fragment thereof, an Outer surface protein C (OspC) polypeptide or an antigenic fragment thereof, an Outer surface protein A (OspA) polypeptide or an antigenic fragment thereof, an Outer surface protein B (OspB) polypeptide or an antigenic fragment thereof, a P100 polypeptide or an antigenic fragment thereof, a P41 polypeptide or an antigenic fragment thereof, a P66 polypeptide or an antigenic fragment thereof, a Borrelia membrane protein A (BmpA) polypeptide or an antigenic fragment thereof, a Borrelia membrane protein B (BmpB) polypeptide or an antigenic fragment thereof, a Borrelia membrane protein C (BmpC) polypeptide or an antigenic fragment thereof, a Borrelia glycosaminoglycan-binding protein (Bgp) polypeptide or an antigenic fragment thereof, and a Fibronectin-binding protein (Fbp) polypeptide or an antigenic fragment thereof.

48. The method of claim 47, wherein the one or more Borrelia antigens are purified recombinant or synthetic polypeptides.

49. The method of claim 47, wherein the one or more Borrelia antigens are a mixture of a DbpA polypeptide or an antigenic fragment thereof, an OspC polypeptide or an antigenic fragment thereof, a P100 polypeptide or an antigenic fragment thereof, and a VlsE polypeptide or an antigenic fragment thereof.

50. The method of claim 47, wherein the one or more Borrelia antigens are a mixture of an OspC polypeptide or an antigenic fragment thereof and a VlsE polypeptide or an antigenic fragment thereof.

51. The method of claim 47, wherein the one or more Borrelia antigens are a mixture of a NapA polypeptide or an antigenic fragment thereof, a DbpA polypeptide or an antigenic fragment thereof, an OspC polypeptide or an antigenic fragment thereof, a P100 polypeptide or an antigenic fragment thereof, and a VlsE polypeptide or an antigenic fragment thereof.

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. A kit for detecting active Borrelia infection, the kit comprising:
a. a solid phase support coated with one or more capture antibodies specific for one or more proteins secreted by Borrelia antigen-specific CD8 effector T cells, and
b. a composition comprising one or more Borrelia antigen polypeptides.

73. The kit of claim 72, wherein the solid phase support is a microwell of a microplate.

74. The kit of claim 72, wherein the one or more proteins secreted by Borrelia antigen-specific CD8 effector T cells are selected from granzyme B, perforin, and granulysin.

75. The kit of claim 72, wherein the one or more Borrelia antigen polypeptides are selected from the group consisting of but not limited to a VlsE polypeptide or an antigenic fragment thereof, a NapA polypeptide or an antigenic fragment thereof, a DbpA polypeptide or an antigenic fragment thereof, a DbpB polypeptide or an antigenic fragment thereof, an OspC polypeptide or an antigenic fragment thereof, an OspA polypeptide or an antigenic fragment thereof, an OspB polypeptide or an antigenic fragment thereof, a P100 polypeptide or an antigenic fragment thereof, a P41 polypeptide or an antigenic fragment thereof, a P66 polypeptide or an antigenic fragment thereof, a BmpA polypeptide or an antigenic fragment thereof, a BmpB polypeptide or an antigenic fragment thereof, a BmpC polypeptide or an antigenic fragment thereof, a Bgp polypeptide or an antigenic fragment thereof, and a Fbp polypeptide or an antigenic fragment thereof.

76. The kit of claim 75, wherein the one or more Borrelia antigen polypeptides are a mixture of a NapA polypeptide or an antigenic fragment thereof, a DbpA polypeptide or an antigenic fragment thereof, an OspC polypeptide or an antigenic fragment thereof, a P100 polypeptide or an antigenic fragment thereof, and a Vlse polypeptide or an antigenic fragment thereof.

77. The kit of claim 75, wherein the one or more Borrelia antigen polypeptides are a mixture of a DbpA polypeptide or an antigenic fragment thereof, an OspC polypeptide or an antigenic fragment thereof, a P100 polypeptide or an antigenic fragment thereof, and a VlsE polypeptide or an antigenic fragment thereof.

78. The kit of claim 75, wherein the one or more Borrelia antigen polypeptides are a mixture of an OspC polypeptide or an antigenic fragment thereof and a VlsE polypeptide or an antigenic fragment thereof.

79. The kit of claim 75, wherein the one or more Borrelia antigen polypeptides are recombinant or synthetic polypeptide.

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. The kit of claim 72, wherein the kit further comprises a detection antibody specific for the one or more proteins secreted by Borrelia antigen-specific CD8 effector T cells.

102. The kit of claim 101, wherein the detection antibody is an enzyme-conjugated antibody.

103. (canceled)

104. (canceled)

105. (canceled)

106. (canceled)

107. (canceled)
